SEQUENCE LISTING (1 sequence)

SEQ ID NO: 1 - protein, 1365 aa, Artificial Sequence
  Synthetic MMSET
MEFSIKQSPLSVQSVVKCIKMKQAPEILGSANGKTPSCEVNRECSVFLSKAQLSSSLQEGVMQKFNGHDALPFIPADKLKDLTSRVFNGEPGAHDAKLRFESQEMKGIGTPPNTTPIKNGSPEIKLKITKTYMNGKPLFESSICGDSAADVSQSEENGQKPENKARRNRKRSIKYDSLLEQGLVEAALVSKISSPSDKKIPAKKESCPNTGRDKDHLLKYNVGDLVWSKVSGYPWWPCMVSADPLLHSYTKLKGQKKSARQYHVQFFGDAPERAWIFEKSLVAFEGEGQFEKLCQESAKQAPTKAEKIKLLKPISGKLRAQWEMGIVQAEEAASMSVEERKAKFTFLYVGDQLHLNPQVAKEAGIAAESLGEMAESSGVSEEAAENPKSVREECIPMKRRRRAKLCSSAETLESHPDIGKSTPQKTAEADPRRGVGSPPGRKKTTVSMPRSRKGDAASQFLVFCQKHRDEVVAEHPDASGEEIEELLRSQWSLLSEKQRARYNTKFALVAPVQAEEDSGNVNGKKRNHTKRIQDPTEDAEAEDTPRKRLRTDKHSLRKRDTITDKTARTSSYKAMEAASSLKSQAATKNLSDACKPLKKRNRASTAASSALGFSKSSSPSASLTENEVSDSPGDEPSESPYESADETQTEVSVSSKKSERGVTAKKEYVCQLCEKPGSLLLCEGPCCGAFHLACLGLSRRPEGRFTCSECASGIHSCFVCKESKTDVKRCVVTQCGKFYHEACVKKYPLTVFESRGFRCPLHSCVSCHASNPSNPRPSKGKMMRCVRCPVAYHSGDACLAAGCSVIASNSIICTAHFTARKGKRHHAHVNVSWCFVCSKGGSLLCCESCPAAFHPDCLNIEMPDGSWFCNDCRAGKKLHFQDIIWVKLGNYRWWPAEVCHPKNVPPNIQKMKHEIGEFPVFFFGSKDYYWTHQARVFPYMEGDRGSRYQGVRGIGRVFKNALQEAEARFREIKLQREARETQESERKPPPYKHIKVNKPYGKVQIYTADISEIPKCNCKPTDENPCGFDSECLNRMLMFECHPQVCPAGEFCQNQCFTKRQYPETKIIKTDGKGWGLVAKRDIRKGEFVNEYVGELIDEEECMARIKHAHENDITHFYMLTIDKDRIIDAGPKGNYSRFMNHSCQPNCETLKWTVNGDTRVGLFAVCDIPAGTELTFNYNLDCLGNEKTVCRCGASNCSGFLGDRPKTSTTLSSEEKGKKTKKKTRRRRAKGEGKRQSEDECFRCGDGGQLVLCDRKFCTKAYHLSCLGLGKRPFGKWECPWHHCDVCGKPSTSFCHLCPNSFCKEHQDGTAFSCTPDGRSYCCEHDLGAASVRSTKTEKPPPEPGKPKGKRRRRRGWRRVTEGK

(19) United States
(12) Patent Application Publication
(10) Pub. No.: US 2021/0189376 A1
(43) Pub. Date: Jun. 24, 2021

(54) CELLS AND METHODS FOR SELECTION BASED ASSAY

(71) Applicant: AMYRIS, INC., Emeryvile, CA (US)

(72) Inventors: Andrew HORWITZ, Emeryvile, CA (US); Jessica Mai WALTER, Emeryvile, CA (US); Chia-Hong TSAI, Emeryville, CA (US)

(21) Appl. No.: 17/271,572

(22) PCT Filed: Aug. 28, 2019
(86) PCT No.: PCT/US2019/048625

§ 371 Date: Feb. 25, 2021

Related U.S. Application Data
(60) Provisional application No. 62/724,231, filed on Aug. 29, 2018.

(51) Int. Cl. C12N 15/10; C12N 15/52

(52) U.S. Cl. CPC C12N 15/102; C12N 15/52

(57) ABSTRACT

Cells and methods for screening inhibitors against a heterologous target protein are disclosed.

### FIELD

Provided are cells and methods for screening inhibitors against a target protein.

### BACKGROUND

High throughput screening for drug discovery typically involves purifying a target protein, developing an in vitro screening assay, and applying purified compound libraries to the assay to identify hits. High throughput screening often relies on robotics, data processing, control software, liquid handling devices, and sensitive detectors. One can rapidly identify active compounds and antibodies that modulate a particular biomolecular pathway with it. High throughput screening allows a researcher to quickly conduct millions of chemical, genetic, or pharmacological tests.

High throughput screening has drawbacks. It is often too expensive to be practical. It sometimes requires pure compounds, which is also not practical under some circumstances. And, high throughput screening is not always perfectly suitable for screening intracellular targets because the cell wall of a cell may be impermeable to compounds and antibodies.

In a biosynthetic library, living cells may be transformed with genes derived from plants, fungi, and bacteria to create randomly assorted metabolic pathways for the production of natural-like chemicals. Over the course of the last few decades, hundreds of natural product biosynthetic pathways and thousands of natural scaffolds, such as peptides, polyketides, terpenoids, and oligosaccharides, have been characterized.

There is a need for screening assays other than traditional high throughput screening assay. For example, a screening assay that is inexpensive and useful at screening heterologous target proteins would be useful. Additionally, a screening assay that can screen biosynthetic libraries would be useful.

### SUMMARY

Provided herein is a screening assay and cells that can be used to screen a target protein that is heterologous to a cell. In the assay, activity of a target protein that is heterologous to the cell is made toxic to the cell through genetic modification or deletion of one or more native genes in the cell. The cell is then exposed to candidate inhibitor compounds. Cells that grow indicate that a potential inhibitor of the target protein has been identified. The method is applicable to the target MMSET expressed in yeast cells.

Cells can be exposed to candidate inhibitor compounds by any method known to one skilled in the art. Exposure of cells to candidate inhibitor compounds may comprise contacting the cells with one or more candidate inhibitor compounds or one or more compound libraries. Cells can also be exposed to candidate inhibitor compounds by expressing a biosynthetic pathway for the candidate inhibitors in the cell.

A first aspect of the invention provides a cell comprising: i) one or more exogenous nucleic acids expressing one or more targets and ii) one or more genes native to the cell genetically modified and/or deleted, wherein the combination of the one or more targets with the genetic modification and/or deletion of one or more genes native to the cell is toxic to the cell. In some embodiments, the combination of the one or more targets with the genetic modification and/or deletion of the one or more genes native to the cell provides a synthetic sick or synthetic lethal interaction to the cell.

Cells can be any of those deemed useful by one skilled in the art. In some embodiments, the cell is selected from the group consisting of archaeal, prokaryotic, or eukaryotic cells. In some embodiments, the cell is a eukaryotic cell. In some embodiments, the cell is a yeast cell. In some embodiments, the yeast cell is Saccharomyces cerevisiae.

In some embodiments, the one or more targets comprises a disease target. In some embodiments, the one or more targets comprises a mammalian target. In some embodiments, the one or more targets comprises a human target. In some embodiments, the disease target comprises a human disease target. In some embodiments, the target comprises any of the targets set forth in this specification.

In some embodiments, the disease target comprises or consists of MMSET. In some embodiments, MMSET comprises or consists of one or more amino acid substitutions from the sequence set forth in SEQ ID NO: 1. In some embodiments, MMSET comprises or consists of one or more of the following substitutions: Y1092A, Y1118A, F1177A, and/or Y1179A, wherein the residue numbers are numbered according to SEQ ID NO: 1. In some embodiments, the one or more targets is one or more MMSET proteins with amino acid substitutions from any of the tables provided herein.

In some embodiments, the modified and/or deleted one or more genes native to the cell are selected from the group consisting of SET2, SWR1, and LGE1. In some embodiments, the modified and/or deleted one or more genes native to the cell comprises or consists of one or both of SET2 and LGE1.

In some embodiments, the cell further comprises one or more nucleic acids encoding enzymes that produce candidate inhibitor compounds. In some embodiments, the one or more nucleic acids encoding enzymes that produce candidate inhibitor compounds comprises one or more metabolic pathways that produce the candidate inhibitor compounds. In some embodiments, the one or more metabolic pathways produce one or more natural compounds or one or more natural-like products. In some embodiments, the one or more nucleic acids encoding enzymes that produce candidate inhibitor compounds comprises nucleic acids derived from plants, fungi, and/or bacteria. In some embodiments, the one or more targets and the one or more nucleic acids encoding enzymes that produce candidate inhibitor compounds are expressed in the same cell.

In some embodiments, the one or more targets comprises a mixture of hyperactive targets and/or catalytically dead targets, the hyperactive targets and/or catalytically dead targets varied in relative abundance to calibrate relative toxicity to the cell. In some embodiments, the mixture of hyperactive targets and/or catalytically dead targets comprises one or more MMSET proteins, each having at least one or more of the following mutations: F1177A, Y1118A, Y1179A, and/or Y1092A, wherein the residues are numbered according to SEQ ID NO: 1.

Another aspect provides a method of detecting inhibitors of one or more targets, comprising:
a) providing a cell comprising one or more exogenous nucleic acids expressing the one or more targets;
b) genetically modifying and/or deleting one or more genes native to the cell, wherein the combination of the one or more targets with the genetic modification and/or deletion of the one or more genes native to the cell is toxic to the cell;
c) exposing the cell to candidate inhibitor compounds;
d) growing the cell under growth conditions; and
e) measuring growth of the cell,
   wherein growth of the cell detects a candidate inhibitor compound as an inhibitor of the one or more targets. In some embodiments, the combination of the one or more targets with genetic modification and/or deletion of the one or more genes native to the cell provides a synthetic sick or synthetic lethal interaction to the cell.

In some embodiments, the cell is selected from the group consisting of archaeal, prokaryotic, or eukaryotic cells. In some embodiments, the cell is a eukaryotic cell. In some embodiments, the cell is a yeast cell. In some embodiments, the yeast cell is Saccharomyces cerevisiae.

In some embodiments, the one or more targets comprises a disease target. In some embodiments, the one or more targets comprises a mammalian target. In some embodiments, the one or more targets comprises a human target. In some embodiments, the disease target comprises a human disease target. In some embodiments, the target comprises any of the targets set forth in this specification.

In some embodiments, the disease target comprises or consists of MMSET. In some embodiments, MMSET comprises or consists of one or more amino acid substitutions from the sequence set forth in SEQ ID NO: 1. In some embodiments, MMSET comprises or consists of one or more of the following substitutions: Y1092A, Y1118A, F1177A, and/or Y1179A, wherein the residue numbers are numbered according to SEQ ID NO: 1. In some embodiments, the one or more targets is one or more MMSET proteins with amino acid substitutions from any of the tables provided herein.

In some embodiments, the modified and/or deleted one or more genes native to the cell are selected from the group consisting of SET2, SWR1, and LGE1 . In some embodiments, the modified and/or deleted one or more genes native to the cell comprises or consists of one or both of SET2 and LGE1.

In some embodiments, exposing the cell to candidate inhibitor compounds comprises expressing in the cell one or more nucleic acids encoding enzymes that produce the candidate inhibitor compounds. In some embodiments, the one or more nucleic acids encoding enzymes that produce candidate inhibitor compounds comprises one or more metabolic pathways that produce the candidate inhibitor compounds. In some embodiments, the one or more metabolic pathways produce one or more natural compounds or one or more natural-like products. In some embodiments, the one or more nucleic acids encoding enzymes that produce candidate inhibitor compounds comprises nucleic acids derived any organism such as, for example, without limitation, from plants, fungi, and/or bacteria.

In some embodiments, exposing the cell to candidate inhibitor compounds comprises contacting the cell with the candidate inhibitor compounds. In some embodiments, contacting the cell comprises adding the candidate inhibitor compounds to a cell culture. In some embodiments, exposing exposure the cell to candidate inhibitor compounds further comprises rendering the cell more permeable to the candidate inhibitor compounds.

In some embodiments, the growth conditions omit one or more of histidine, uracil, and/or lysine.

In some embodiments, the growth conditions comprise growing the cell at a temperature of less than about 30° C. In some embodiments, the growth conditions comprise growing the cell at a temperature of less than about 29° C. In some embodiments, the growth conditions comprise growing the cell at a temperature of less than about 28° C. In some embodiments, the growth conditions comprise growing the cell at a temperature of less than about 27° C. In some embodiments, the growth conditions comprise growing the cell at a temperature of less than about 26° C. In some embodiments, the growth conditions comprise growing the cell at a temperature of less than about 25° C. In some embodiments, the growth conditions comprise growing the cell at a temperature of less than about 24° C. In some embodiments, the growth conditions comprise growing the cell at a temperature of less than about 23° C. In some embodiments, the growth conditions comprise growing the cell at a temperature of less than about 22° C. In some embodiments, the growth conditions comprise growing the cell at a temperature of less than about 21° C. In some embodiments, the growth conditions comprise growing the cell at a temperature of less than about 20° C.

Any method known to one skilled in the art may be used to measure growth of the cell or colony size. A cell viability assay may be used to measure cell growth. A cell viability assay may be used to measure colony size. Cellular growth may also measured using foci formation screens, nuclear and cellular morphology screens, and localization of proteins. Reporter gene assay screens may also be used. Compound screens may utilize cells plated in 96 or 384 well plates to produce a visual phenotypic change in the cells that can be quantified. In some embodiments, measuring growth of the cell comprises calculating population size using a Z-factor or Hedge's effect.

In some embodiments, the one or more targets comprises a mixture of hyperactive targets and/or catalytically dead targets, the hyperactive targets and/or catalytically dead targets varied in relative abundance to calibrate relative toxicity to the cell. In some embodiments, the mixture of hyperactive targets and/or catalytically dead targets comprises one or more MMSET proteins, each having at least one or more of the following mutations: F1177A, Y1118A, Y1179A, and/or Y1092A, wherein the residues are numbered according to SEQ ID NO: 1. Catalytically dead targets simulate successful inhibition by an exogenously added or internally produced compound.

### BRIEF DESCRIPTION OF THE FIGURES

FIG. 1 depicts an assay that can be used to screen thousands of molecules against a target in a cell.

FIG. 2 depicts results from a hypothetical relief screen (FIG. 2A) and an assay for selection based upon relief from MMSET toxicity (FIG. 2B)

FIG. 3 depicts an epistasis map.

FIG. 4 depicts the mildly toxic effect of overexpression of MMSET in yeast (FIG. 4A) and additional catalytically dead mutants that rescue MMSET (FIG. 4B)

FIG. 5 depicts SET 2 deletion combined with knockouts of other genes and MMSET overexpression in set2A, lgeA strain backgrounds.

FIG. 6 depicts MMSET-FY (catalytically dead, left) and MMSET-F (hyperactive, right) colony sizes when plated on media depleted of histidine, uracil, and/or lysine.

FIG. 7 depicts an equal mixture of LGE knockout large (MMSET-FY) and small (MMSET-F) colonies plated, scanned, and measured (left) and a histogram of measured colonies (right).

FIG. 8 depicts cells with increasingly large fractions of inhibited MMSET that produce progressively larger colonies in a ALGE1 background.

FIG. 9 depicts di-methylated histone 3 at lysine 36 (H3K36me2) in wild-type strains and SET2 knockout strains with MMSET variants.

FIG. 10 depicts growth of ASET2 ALGE1 MMSET yeast strains at three temperatures.

FIG. 11 depicts combinatorial transformation of diterpene synthases, P450s, and hydroxyl-modifying enzymes.

FIG. 12 depicts a distribution of enzymes in a random sampling. FIG. 12A depicts the distribution of enzymes of a random sampling of 192 colonies in the production strain library. FIG. 12B depicts a distribution of enzymes in a random sampling of 96 production strain colonies transformed with the small library.

FIG. 13 depicts dual column GC-FID traces of single colonies from the production strain library show great diversity in peak distribution from the parent strain.

FIG. 14 depicts colony size growth rate verification.

FIG. 15 depicts two colonies with potentially inhibited MMSET isolated from library transformation.

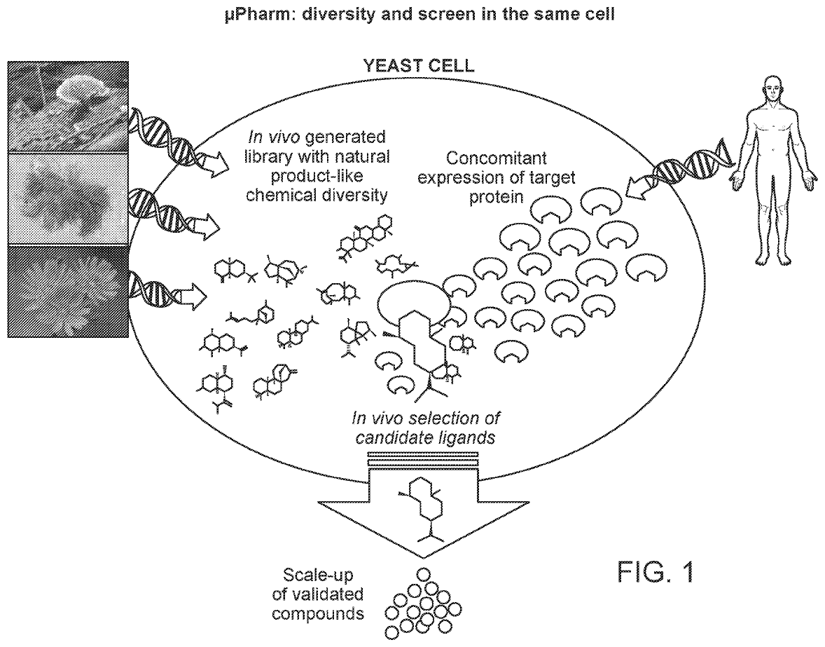

### DESCRIPTION OF EXEMPLARY EMBODIMENTS

Provided herein are methods and cells that can be used in those methods. In particular, activity of a heterologous target is made toxic to a cell through genetic modification or deletion of a gene in the cell. Engineered toxicity retards growth of the cell until the cell is rescued through exposure to an inhibitor of the heterologous target. The method is considered to have identified an inhibitor of the target when the cell grows.

One particular advantage is that the method is well suited to screening biosynthetic libraries, such as biosynthetic libraries where the compounds or compound libraries are expressed in the cell. In the biosynthetic library approach, living cells are transformed with genes derived from plants, fungi, and bacteria to create metabolic pathways for production of diverse natural compounds or natural-like compounds. If the assay cell is transformed with a biosynthetic library that rescues the cell, the cell will form growing colonies. This allows screening of massive genetic libraries without handling individual clones or purifying individual compounds.

Another advantage is that the assay can be inexpensive as the assay involves a self-replicating microbial cell. Another advantage is that efficacy can be measured simply by measuring colony sizes.

A non-limiting example provided herein is a yeast cell that expresses MMSET with deletion of the gene that is orthologous to MMSET in yeast, SET2. MMSET is a histone methyltransferase implicated in multiple myeloma in humans. When MMSET was expressed in the yeast with a deletion of SET2, a mild growth defect was observed as a toxic phenotype.

To amplify the toxic phenotype, a series of additional deletions thought to have a synthetic sick effect in yeast in combination with expression of hyperactive MMSET and deletion of SET2 were identified, including the LGE1 gene. A deletion of LGE1 was incorporated into the method to further amplify the toxic phenotype.

The method could then be used to detect inhibitors of MMSET. For example, when an inhibitor of MMSET was added to the cell, the cell responded to the inhibitor by growing more rapidly and forming larger colonies.

### 1. Definitions

When referring to the compositions and methods provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term herein, those in this section prevail unless stated otherwise.

As used herein, “candidate gene approach” refers to association studies conducted to focus on genetic variation within a set of pre-specified genes of interest and phenotypes or disease states.

As used herein, a “compound library” or “chemical library” refers to a collection of stored chemicals. Some embodiments are drawn to compound libraries. The compound library or chemical library can consist simply of stored chemicals or the compound library may be encoded on one or more nucleic acids.

As used herein, “conservative amino acid substitution” refers to a substitution in which an amino acid residue is substituted by another amino acid residue having a side chain (R group) with similar chemical properties (e.g., charge or hydrophobicity). In general, a conservative amino acid substitution should not substantially change the functional properties of a protein. The following six groups each contain amino acids that are often, depending upon context, considered conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Alanine (A), Valine (V), and 6) Phenylalanine (F), Tyrosine (Y), Tryptophan (W).

As used herein, “enzyme” or “enzymatically” refers to biological catalysts. Enzymes accelerate, or catalyze, chemical reactions. Like all catalysts, enzymes increase the rate of reaction by lowering the activation energy. In some embodiments, the target is an enzyme. The term enzyme may also refer to a protein capable of making, or catalyzing a step in the making of, candidate inhibitor compounds or inhibitor compounds, as set forth herein.

As used herein, the term “epistasis” or “epistatic” refers to the suppression or enhancement of one genetic alteration on another. In particular, epistasis refers to the suppression of the effect of one such gene by another.

As used herein, “exogenous” refers to something, such as a gene or polynucleotide, that originates outside of an organism of concern or study. An exogenous polynucleotide, for example, may be introduced into a cell or organism by introduction into the cell or organism of an encoding nucleic acid. Exogenous expression of an encoding nucleic acid can utilize either or both a heterologous or homologous encoding nucleic acid. A nucleic acid need not include all of its relevant or even complete coding regions on a single nucleic acid and in some embodiments, complete or partial coding sequences are provided on different nucleic acids.

As used herein, “exposed” or “exposing” refers to subjecting cells or one or more targets to candidate inhibitor compounds. Exposure may occur by any means known to one skilled in the art.

As used herein, “genetic alteration,” “genetically altered,” “genetic engineering,” “genetically engineered,” “genetic modification,” “genetically modified,” “genetic regulation,” or “genetically regulated” shall be used interchangeably and refer to direct or indirect manipulation of an organism's genome or genes to produce, for example, a desired effect, such as a desired phenotype. Genetic alteration includes a set of technologies that can be used to change genetic makeup, which ultimately could lead to the suppression or enhancement of phenotype or expression of a gene, as used herein. Genetic alteration shall also include the ability to reduce or prevent expression of a gene or genes. Genetic alteration techniques shall include, for example, molecular cloning, gene knockouts, gene targeting, mutation, homologous recombination, gene deletion, gene knockdown, gene silencing, gene addition, genome editing, gene attenuation, or any technique that may be used to suppress or alter the expression of a gene and a phenotype.

As used herein, “gene deletion” or “deletion” refers to a mutation or genetic modification in which a sequence of DNA is lost, deleted, or modified. A gene may be deleted to alter a cell's genome or to produce a desired effect or desired phenotype.

As used herein, “gene knockdown” refers to a technique by which expression of one or more genes are reduced. Reduction can occur by any method known to one skilled in the art such as genetic modification or by treatment with a reagent such as a short DNA or RNA oligonucleotide that has a sequence complimentary to either a gene or an mRNA transcript.

As used herein, “gene knockout” refers to a procedure whereby a gene is made inoperative.

As used herein, “gene silencing,” “silencing,” or “silenced” refers to the regulation of a gene, in particular, the down regulation of a gene. Specifically, the term refers to the ability to reduce or prevent the expression of a certain gene. Gene silencing can occur at any cellular process, such as during transcription or translation. Any methods of gene silencing well known in the art may be used.

As used herein, “homology” or “homologous” refers to sequence homology, the biological homology between protein or polynucleotide sequences with respect to shared ancestry as determined by the closeness of nucleotide or protein sequences. Homology among proteins or polynucleotides is typically inferred from their sequence similarity. Alignments of multiple sequences are used to indicate which regions of each sequence are homologous. The term “percent homology” refers to the percentage of identical residues (percent identity) or the percentage of residues conserved with similar physiochemical properties (percent similarity) and is usually used to quantify homology.

As used herein, “metabolic pathway” refers to a linked series of chemical reactions occurring within a cell. Reactants, products, and intermediates of an enzymatic reaction are modified by a sequence of chemical reactions catalyzed by enzymes. In a metabolic pathway, a product of one enzyme acts as the substrate for the next.

As used herein, a “natural compound” or “natural product” refers to a chemical compound or substance produced by a living organism. In the broadest sense, natural compounds or natural products include any substance produced by something that is alive. Natural products may be prepared by chemical synthesis.

As used herein, “natural-like compounds,” “natural-like products,” or “natural product-like” refers to compounds that have properties that are similar or identical to natural compounds. Natural-like compounds can be selected according to their similarity to natural compounds.

As used herein, “screening approach,” “genetic screen,” “genetic screen approach,” or “mutagenesis screening approach” refers to a technique used to identify and select for organisms that possess a phenotype of interest in a mutagenized population. A genetic screen is a type of phenotypic screen. Genetic screens can provide important information on gene function as well as the molecular events that underlie a biological process or pathway.

As used herein, “synthetic lethal” refers to a non-viable phenotype that results from genetic alterations.

As used herein, “synthetic sick” refers to a phenotype that is viable but that has lower fitness than a wild type.

As used herein, “target,” “biological target,” or “drug target” refers to a molecule, such as a native protein, or a portion of the protein thereof as provided herein, which molecule has activity and such activity may be modified by an inhibitor resulting in a specific effect. A target may be used for a desirable effect or an unwanted adverse effect. An example of a target is MMSET, a histone methyltranferase whose overexpression and misregulation is associated with multiple myeloma. Inhibition of the activity of MMSET could have a therapeutic effect for a patient in need.

As used herein, “toxic” refers to an interaction that kills, injures, or impairs a cell. Toxic also refers to an epistatic relationship that produces a synthetic sick or synthetic lethal phenotype.

As used herein, “Z-factor” or “Hedges' Effect Size” refers to a measure of statistical effect size.

### 2. Methods and Cells

A first aspect of the invention provides a cell comprising: i) one or more exogenous nucleic acids expressing one or more targets and ii) one or more genes native to the cell genetically modified and/or deleted, wherein the combination of the one or more targets with the genetic modification and/or deletion of one or more genes native to the cell is toxic to the cell. In some embodiments, the combination of the one or more targets with the genetic modification and/or deletion of the one or more genes native to the cell provides a synthetic sick or synthetic lethal interaction to the cell.

In some embodiments, the one or more genes native to the cell comprises genes native to the cell that are homologous or orthologous to the exogenous nucleic acids encoding the one or more targets. In some embodiments, the one or more genes native to the cell are identified with a candidate gene approach. With respect to the MMSET target, a candidate gene approach was taken by searching the Krogan lab database of genetic interactions to identify a set of genes that had interaction with the yeast orthologue of MMSET (See, for example, www.interactome-cmp.ucsfedu, which is incorporated by reference in its entirety herein) and the SET2 gene was identified. SET2 also contains conserved protein domains also contained within MMSET. Genetic interactions of SET2 with other genes (SWR1 and LGE1) were identified from the database.

In some embodiments, the one or more gene native to the cell are identified with a screening approach. For example, a library based approach could be easily undertaken using standard E-MAP techniques (See, for example, Collins S., Roguev, A., and Krogan N., Quantitative Genetic Interaction Mapping Using the E-Map Approach, Methods Enzymol. 2010; 470: 205-231, which is incorporated by reference in its entirety herein, including any drawings).

In some embodiments, the modified and/or deleted one or more genes native to the cell are selected from the group consisting of SET2, SWR1, and LGE1 . In some embodiments, the modified and/or deleted one or more genes native to the cell comprises or consists of one or both of SET2 and LGE1.

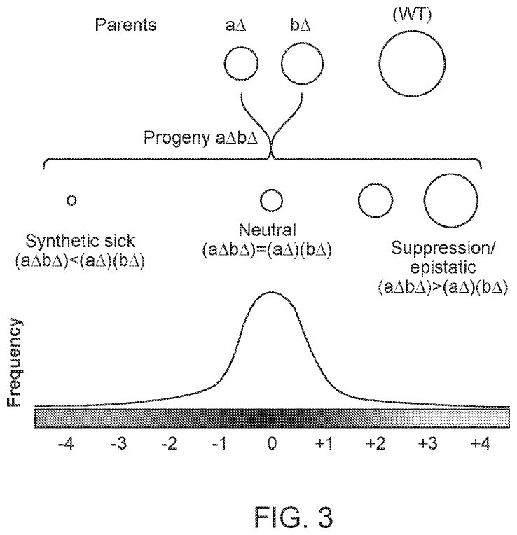

The combination of the expression of the one or more exogenous nucleic acids with the genetic modifications of one or more genes native to the cell and/or a deletion of one or more genes native to the cell may produce epistasis in the cell. Epistasis is the suppression or enhancement of a cell phenotype through one genetic alteration as it relates to another. In epistasis, the effect of modifying or deleting one gene is amplified or suppressed by modification or deletion of a second gene. Epistasis can be studied in high throughput by use of epistasis maps (E-Maps) that combine modifications or deletions of genes and measure colony size as a proxy for “fitness.” An epistasis map is depicted in FIG. 3, which shows that quantitative genetic analysis can identify negative ((aΔbΔ)<(aΔ) (bΔ)), positive ((aΔbΔA)>(aΔ) (bΔ)), and neutral ((aΔbΔ)=(aΔ)(bΔ)) genetic interactions.

For non-interacting genes, colony size should be the product of the fractions of wild-type colony size. For examples, two mutations that each give a colony size 0.5 of WT should give colony size of 0.25 when combined. Deviations from this represent synthetic effects, or epistasis. Suppression usually occurs when the two modified or deleted genes are in the same functional pathway, i.e., the damage is fully realized by modifying or deleting one, and modification or deletion of the second is redundant. Synthetic sick effects usually occur when the two modified or deleted genes are in complementary pathways, e.g., two separate pathways that address the same cellular need. In such a case, incapacitating both pathways has a synthetic, negative effect on the cell.

While epistasis usually refers to interactions between native genes (i.e. genetic modifications and/or deletions of those genes), epistasis may also apply to heterologous genes or a heterologous gene and a native gene. For example, native genes homologous or orthologous to a heterologous target may be genetically modified and/or deleted from the native cell to increase the efficiency of the method. Other genes native to the cell may be modified and/or deleted to increase efficiency of the method.

Toxicity will severely retard growth of the synthetically sick cell until the cell is rescued by exposing the heterologous enzyme to an inhibitor of the target. The inhibitor will allow the cell to grow, thus confirming that the inhibitor is an inhibitor of the heterologous target.

### 3. Useful Cells

Cells that can be used may be any cells deemed useful by those of skill in the art. Cells useful in the compositions and methods provided herein include archaeal, prokaryotic, or eukaryotic cells.

In some embodiments, the cells are prokaryotic cells. In some embodiments, the cells are any one of gram-positive, gram-negative, or gram-variable bacteria. Examples include, but are not limited to, cells belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphlococcus, Strepromyces, Synnecoccus, and Zymomonas. Examples of strains include, but are not limited to: Bacillus subtilis, Bacillus amyloliquefacines, Brevibacterium ammoniagenes, Brevibacterium immariophilum, Clostridium beigerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus.

In some embodiments, the cells are archaeal cells. In some embodiments, archaeal cells include, but are not limited to: Aeropyrum, Archaeglobus, Halobacterium, Methanococcus, Methanobacterium, Pyrococcus, Sulfolobus, and Thermoplasma. Examples of archaea strains include, but are not limited to: Archaeoglobus fulgidus, Halobacterium sp., Methanococcus jannaschii, Methanobacterium thermoautotrophicum, Thermoplasma acidophilum, Thermoplasma volcanium, Pyrococcus horikoshii, Pyrococcus abyssi, and Aeropyrum pernix.

In some embodiments, the cells are eukaryotic cells. In some embodiments, the eukaryotic cells include, but are not limited to, fungal cells, algal cells, insect cells, and plant cells. In some embodiments, yeasts useful in the present methods include yeasts that have been deposited with microorganism depositories (e.g. IFO, ATCC, etc). and belong to the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, Schizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In some embodiments, the cell is Saccharomyces cerevisiae, Pichia pastoris, Schizosaccharomyces pombe, Dekkera bruxellensis, Kluyveromyces lactis (previously called Saccharomyces lactis), Kluveromyces marxianus, Arxula adeninivorans, or Hansenula polymorpha (now known as Pichia angusta). In some embodiments, the cell is a strain of the genus Candida, such as Candida lipolytica, Candida guilliermondii, Candida krusei, Candida pseudotropicalis, or Candida utilis.

In some embodiments, the cell is Saccharomyces cerevisiae. In some embodiments, the cell is a strain of Saccharomyces cerevisiae selected from the group consisting of Baker's yeast, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1, BR-1, BR-2, ME-2, VR-2, MA-3, MA-4, CAT-1, CB-1, NR-1, BT-1, and AL-1. In some embodiments, the host cell is a strain of Saccharomyces cerevisiae selected from the group consisting of PE-2, CAT-1, VR-1, BG-1, CR-1, CEN.PK113-7D, CEN.PK2, and SA-1. In some embodiments, the strain of Saccharomyces cerevisiae is PE-2. In another some embodiments, the strain of Saccharomyces cerevisiae is CAT-1. In some embodiments, the strain of Saccharomyces cerevisiae is BG-1. In some embodiments, the strain of Saccharomyces cerevisiae is that created and set forth in the examples herein.

In some embodiments, the cell is a microbe. In some embodiments, the microbe is conditioned to subsist under high solvent concentration, high temperature, expanded substrate utilization, nutrient limitation, osmotic stress due to sugar and salts, acidity, sulphite, and bacterial contamination, or combinations thereof, which are recognized stress conditions of the industrial fermentation environment.

### 4. Exposure to Candidate Inhibitor Compounds

Cells can be exposed to candidate inhibitor compounds by any method known to one skilled in the art. Exposure of cells to candidate inhibitor compounds may comprise, for example, without limitation, contacting the cells with one or more candidate inhibitor compounds or one or more compound libraries. In some embodiments, contacting the cell comprises adding the one or more candidate inhibitor compounds to a cell culture.

In some embodiments, exposing the cell to candidate inhibitor compounds further comprises rendering the cell more permeable to the candidate inhibitor compounds. Any method of making the cells more permeable to candidate inhibitor compounds known to one skilled in the art may be used (See, for example, Pannunzio V. G., Burgos, M., Alonso, J. R., Ramos, E. H., and Stella, C. A. (2004,) A Simple Chemical Method for Rendering Wild-Type Yeast Permeable to Brefeldin A that does not Require the Presence of an erg6 Mutation J. Biomed. Biotechnol. 150-155, which is incorporated by reference in its entirety herein, including any drawings).

Cells can also be exposed to candidate inhibitor compounds when cells are transformed with an inhibitor library to produce inhibitors. The library may be a biosynthetic library with genes derived from plants, fungi, and bacteria. The library may be a biosynthetic library with genes derived from plants, fungi, and bacteria that creates randomly assorted metabolic pathways for production of diverse natural compounds or natural-like compounds. Only cells that can make inhibitors of the one or more targets will grow and form colonies.

In some embodiments, exposing the cell to candidate inhibitor compounds comprises expressing in the cell one or more nucleic acids encoding enzymes that produce the candidate inhibitor compounds. In some embodiments, the one or more nucleic acids encoding enzymes that produce candidate inhibitor compounds comprises one or more metabolic pathways that produce the candidate inhibitor compounds. In some embodiments, the one or more metabolic pathways produce one or more natural compounds or one or more natural-like products. In some embodiments, the one or more nucleic acids encoding enzymes that produce candidate inhibitor compounds comprises nucleic acids derived from plants, fungi, and/or bacteria.

In some embodiments, the one or more nucleic acids encoding enzymes that produce candidate inhibitor compounds comprises one or more nucleic acids comprising one or more enzymes capable of making candidate inhibitor compounds. In some embodiments, the one or more enzymes are from an anabolic pathway and are capable of making an anabolic product. The anabolic pathway can be any anabolic pathway deemed useful by the practitioner of skill. In some embodiments, the pathway is selected from the group consisting of isoprenoid pathways, polyketide pathways, and fatty acid pathways. Those of skill in the art will recognize that the isoprenoid pathways are capable of making one or more isoprenoid compounds. The polyketide pathways are capable of making one or more polyketide compounds. The fatty acid pathways are capable of making one or more fatty acids. The one or more nucleic acids can comprise enzymes of one pathway or more than one pathway.

In some embodiments, the one or more enzymes further comprise or consist of one or more of terpene synthases, P450 monooxyganases and/or associated redox partners, and hydroxyl-modifying enzymes. In some embodiments, the enzymes further comprise one or more of the enzymes in Table 4 and/or Table 6. Those of skill can select those enzymes that make the final product of a pathway or they can select a subset of the enzymes to make an intermediate product of a pathway. Enzymes can comprise all of the enzymes of a pathway or only a subset of the enzymes of a pathway.

Candidate inhibitor compounds can be any molecule known to one skilled in the art. In some embodiments, candidate inhibitor compounds comprise anabolic compounds. In some embodiments, candidate inhibitor compounds comprise isoprenoid compounds. In some embodiments, candidate inhibitor compounds comprise polyketide compounds. In some embodiments, candidate inhibitor compounds comprise terpene compounds. In some embodiments, candidate inhibitor compounds comprise one or more fatty acids. In some embodiments, candidate inhibitor compounds comprise peptides. In some embodiments, candidate inhibitor compounds comprise oligosaccharides. In some embodiments, candidate inhibitor compounds comprise small molecules.

### 5. Targets

In some embodiments, the one or more targets comprises a disease target. In some embodiments, the one or more targets comprises a mammalian target. In some embodiments, the one or more targets comprises a human target. In some embodiments, the disease target comprises a human disease target. In some embodiments, the one or more targets comprises any of the targets set forth in this specification.

A target selected for the method can be any target deemed useful by one skilled in the art. In some embodiments, the one or more targets is an intracellular protein. In some embodiments, the one or more targets is a receptor. In some embodiments, the one or more targets is a signalling molecule. In some embodiments, the one or more targets is a protein. In some embodiments, the one or more targets is a soluble protein. In some embodiments, the one or more targets is a membrane protein. In some embodiments, the one or more targets is a nuclear receptor. In some embodiments, the one or more targets is a mammalian protein. In some embodiments, the one or more targets is an animal protein. In some embodiments, the one or more targets is a human protein.

In some embodiments, the one or more targets comprises an entire target. In some embodiments, the one or more targets comprises a portion of a target. The portion can be a subunit of a target or a domain of a target. For instance, in some embodiments, the one or more targets comprises a substrate binding domain or subunit of a target. In some embodiments, the one or more targets comprises a nucleic acid binding domain or subunit of a target. In some embodiments, the one or more targets comprises a membrane-binding domain or subunit of a target. In some embodiments, the one or more targets comprises a cofactor-binding domain or subunit of a target. In some embodiments, the one or more targets comprises an allosteric domain or subunit of a target.

In some embodiments, the one or more targets comprises one or more intracellular targets or proteins or one or more targets, proteins, or enzymes inside the cell. The amount of protein in cells is extremely high and approaches 200 mg/ml, occupying about 20-30% of the volume of the cell. Some embodiments of the invention provide a cell comprising one or more targets expressed in the cell with one or more nucleic acids encoding candidate inhibitor compounds. Where the one or more targets are one or more intracellular targets, candidate inhibitors expressed in the same cell as the one or more targets will be able to contact the one or more targets more readily.

In some embodiments, the one or more targets may include, but not be limited to, receptors (e.g., cytokine receptors, immunoglobulin receptors, ligand-gated ion channels, protein kinase receptors, G-protein coupled receptors (GPCRs) nuclear hormone receptors, and other receptors), signalling molecules (e.g., cytokines, growth factors, peptide hormones, chemokines, membrane-bound signalling molecules, and other signalling molecules), kinases (e.g., amino acid kinases, carbohydrate kinases, nucleotide kinases, protein kinases, and other kinases), phosphatases (e.g., carbohydrate phosphatases, nucleotide phosphatases, protein phosphatases, and other phosphatases), proteases (e.g., aspartic proteases, cysteine proteases, metalloproteases, serine proteases, and other proteases), regulatory molecules (e.g., G-protein modulators, large G-proteins, small GTPases, kinase modulators, phosphatase modulators, protease inhibitors, and other enzyme regulators), calcium binding proteins (e.g., annexins, calmodulin related proteins, and other select calcium binding proteins), transcription factors (e.g., nuclear hormone receptors, basal transcription factors, basic helix-loop-helix transcription factors, creb transcription factors, HMG-box transcription factors, homeobox transcription factors, other transcription factors, transcription cofactors, and zinc finger transcription factors), nucleic acid binding proteins (e.g., helicases, DNA ligases, DNA methyltransferases, RNA methyltransferases, double-stranded DNA binding proteins, endodeoxyribonucleases, replication origin binding proteins, reverse transcriptases, ribonucleoproteins, ribosomal proteins, single-stranded DNA-binding proteins, centromere DNA-binding proteins, chromatin/chromatin-binding proteins, DNA glycosylases, DNA photolyases, DNA polymerase processivity factors, DNA strand-pairing proteins, DNA topoisomerases, DNA-directed DNA polymerases, DNA-directed RNA polymerases, damaged DNA-binding proteins, histones, primases, endoribonucleases, exodeoxyribonucleases, exoribonucleases, translation elongation factors, translation initiation factors, translation release factors, mRNA polyadenylation factors, mRNA splicing factors, other DNA-binding proteins, other RNA-binding proteins, and other nucleic acid binding proteins), ion channels (e.g., anion channels, ligand-gated ion channels, voltage-gated ion channels, and other ion channels), transporters (e.g., cation transporters, ATP-binding cassette (ABC) transporters, amino acid transporters, carbohydrate transporters, and other transporters), transfer/carrier proteins (e.g., apolipoproteins, mitochondrial carrier proteins, and other transfer/carrier proteins), cell adhesion molecules (e.g., CAM family adhesion molecules, cadherins, and other cell adhesion molecules), cytoskeletal proteins (e.g., actin and actin related proteins, actin binding motor proteins, non-motor actin binding proteins, other actin family cytoskeletal proteins, intermediate filaments, microtubule family cytoskeletal proteins, and other cytoskeletal proteins), extracellular matrices (e.g., extracellular matrix glycoproteins, extracellular matrix linker proteins, extracellular matrix structural proteins, and other extracellular matrices), cell junction proteins (e.g., gap junction proteins, tight junction proteins, and other cell junction proteins), synthases, synthetases, oxidoreductases (e.g., dehydrogenases, hydroxylases, oxidases, oxygenases, peroxidases, reductases, and other oxidoreductases), transferases (e.g., methyltransferases, acetyltransferases, acyltransferases, glycosyltransferases, nucleotidyltransferases, phosphorylases, transaldolases, transaminases, transketolases, and other transferases), hydrolyases (e.g., deacetylases, deaminases, esterases, galactosidases, glucosidases, glycosidases, lipases, phosphodiesterases, pyrophosphatases, amylases, and other hydrolases), lysases (e.g., adenylate cyclases, guanylate cyclases, aldolases, decarboxylase,s dehydratases, hydratases, and other lyases), isomerases (e.g., epimerase/racemases, mutases, and other isomerases), ligases (e.g., DNA ligases, ubiquitin-protein ligases, and other ligases), defense/immunity proteins (e.g., antibacterial response proteins, complement components, immunoglobulins, immunoglobulin receptor family members, major histocompatibility complex antigens, and other defense and immunity proteins), membrane traffic proteins (e.g., membrane traffic regulatory proteins, SNARE proteins, vesicle coat proteins, and other membrane traffic proteins), chaperones (e.g., chaperonins, hsp 70 family chaperones, hsp 90 family chaperones, and other chaperones), viral proteins (e.g., viral coat proteins and other viral proteins), bacterial proteins, myelin proteins, other miscellaneous function proteins, storage proteins, structural proteins, surfactants, and transmembrane receptor regulatory/adaptor proteins. Other examples of proteins and their functions include those identified in Thomas et al., 2003, Genome Res. 13: 2129-2141, which is incorporated herein by reference in its entirety.

In some embodiments, the target is MMSET. MMSET (multiple myeloma SET domain) is a histone methyltransferase whose overexpression and misregulation is associated with the blood cancer multiple myeloma. As a result, specific inhibitors of MMSET catalytic activity have the potential for therapeutic benefit. Currently, there is no known inhibitor of MMSET.

In some embodiments, MMSET comprises or consists of one or more amino acid substitutions from the sequence set forth in SEQ ID NO: 1. In some embodiments, MMSET comprises or consists of one or more of the following substitutions: Y1092A, Y1118A, F1177A, and/or Y1179A, wherein the residue numbers are numbered according to SEQ ID NO: 1. In some embodiments, the one or more targets is one or more MMSET proteins with amino acid substitutions from any of the tables provided herein.

### 6. Expressing Nucleic Acids in Cells

A first aspect of the invention provides a cell comprising one or more exogenous nucleic acids. In some embodiments, the one or more exogenous nucleic acids are expressed in the cell. Expression of one or more exogenous nucleic acids in a cell can be accomplished by introducing into the cell a nucleic acid comprising a nucleotide sequence encoding the one or more targets under the control of regulatory elements that permit expression in the cell.

Nucleic acids encoding one or more targets can be introduced into a cell by any method known to one of skill in the art (See, for example, Hinnen et al. (1978) Proc. Natl. Acad. Sci. USA 75:1292-3; Cregg et al. (1985) Mol. Cell. Biol. 5:3376-3385; Goeddel et al. eds, 1990, Methods in Enzymology, vol. 185, Academic Press, Inc., CA; Krieger, 1990, Gene Transfer and Expression—A Laboratory Manual, Stockton Press, NY; Sambrook et al., 1989, Molecular Cloning—A Laboratory Manual, Cold Spring Harbor Laboratory, NY; and Ausubel et al., eds., Current Edition, Current Protocols in Molecular Biology, Greene Publishing Associates and Wiley Interscience, NY, each of which is incorporated by reference in its entirety herein, including any drawings). Exemplary techniques include, but are not limited to, spheroplasting, electroporation, PEG 1000 mediated transformation, and lithium acetate or lithium chloride mediated transformation. In some embodiments, the nucleic acid is an extrachromosomal plasmid. In some embodiments, the nucleic acid is a chromosomal integration vector that can integrate the nucleotide sequence into the chromosome of the cell.

Expression of genes may be modified. In some embodiments, expression of the one of more exogenous nucleic acids is modified. For example, the copy number of the one or more exogenous nucleic acids encoding one or more targets in a cell may be altered by modifying the transcription of the gene that encodes the one or more targets. This can be achieved, for example, by modifying the copy number of the nucleotide sequence encoding the one or more targets (e.g., by using a higher or lower copy number expression vector comprising the nucleotide sequence, or by introducing additional copies of the nucleotide sequence into the genome of the cell or by genetically modifying or deleting or disrupting the nucleotide sequence in the genome of the cell), by changing the order of coding sequences on a polycistronic mRNA of an operon, or by breaking up an operon into individual genes, each with its own control elements. The strength of the promoter, enhancer, or operator to which the nucleotide sequence is operably linked may also be manipulated, increased, decreased, or different promoters, enhancers, or operators may be introduced.

Alternatively, or in addition, the copy number of one or more nucleic acids may be altered by modifying the level of translation of an mRNA that encodes the one or more targets. This can be achieved, for example, by modifying the stability of the mRNA, modifying the sequence of the ribosome binding site, modifying the distance or sequence between the ribosome binding site and the start codon of the enzyme coding sequence, modifying the entire intercistronic region located “upstream of” or adjacent to the 5′ side of the start codon of the enzyme coding region, stabilizing the 3′-end of the mRNA transcript using hairpins and specialized sequences, modifying the codon usage of an enzyme, altering expression of rare codon tRNAs used in the biosynthesis of the enzyme, and/or increasing the stability of an enzyme, as, for example, via mutation of its coding sequence.

Expression of the one or more exogenous nucleic acids may be modified or regulated by targeting particular sequences. For example, the cell may be contacted with one or more nucleases capable of cleaving, i.e., causing a break at a designated region within a selected site. In some embodiments, the break is a single-stranded break, that is, one but not both strands of a site is cleaved. In some embodiments, the break is a double-stranded break. In some embodiments, a break inducing agent, any agent that recognizes and/or binds to a specific polynucleotide recognition sequence to produce a break at or near a recognition sequence, is used. Examples of break inducing agents include, but are not limited to, endonucleases, site-specific recombinases, transposases, topoisomerases, and zinc finger nucleases, and include modified derivatives, variants, and fragments thereof

In some embodiments, the recognition sequence within a selected site can be endogenous or exogenous to a cell's genome. When the recognition site is an endogenous or exogenous sequence, it may be a recognition sequence recognized by a naturally occurring or native break inducing agent. Alternatively, an endogenous or exogenous recognition site could be recognized and/or bound by a modified or engineered break inducing agent designed or selected to specifically recognize the endogenous or exogenous recognition sequence to produce a break. In some embodiments, the modified break inducing agent is derived from a native, naturally occurring break inducing agent. In other embodiments, the modified break inducing agent is artificially created or synthesized. Methods for selecting such modified or engineered break inducing agents are known in the art.

In some embodiments, the one or more nucleases is a CRISPR/Cas-derived RNA-guided endonuclease. CRISPR may be used to recognize, genetically modify, and/or silence genetic elements at the RNA or DNA level or to express heterologous or homologous genes. CRISPR may also be used to regulate endogenous or exogenous nucleic acids. Any CRISPR/Cas system known in the art finds use as a nuclease in the methods and compositions provided herein. CRISPR systems that find use in the methods and compositions provided herein also include those described in International Publication Numbers WO 2013/142578 A1, WO 2013/098244 A1 and Nucleic Acids Res (2017) 45 (1): 496-508, the contents of which are hereby incorporated in their entireties).

In some embodiments, the one or more nucleases is a TAL-effector DNA binding domain-nuclease fusion protein (TALEN). TAL effectors of plant pathogenic bacteria in the genus Xanthomonas play important roles in disease, or trigger defence, by binding host DNA and activating effector-specific host genes. (See, e.g., Gu et al. (2005) Nature 435:1122-5; Yang et al., (2006) Proc. Natl. Acad. Sci. USA 103:10503-8; Kay et al., (2007) Science 318:648-51; Sugio et al., (2007) Proc. Natl. Acad. Sci. USA 104:10720-5; Romer et al., (2007) Science 318:645-8; Boch et al., (2009) Science 326(5959):1509-12; and Moscou and Bogdanove, (2009) 326(5959):1501, each of which is incorporated by reference in their entirety). A TAL effector comprises a DNA binding domain that interacts with DNA in a sequence-specific manner through one or more tandem repeat domains. The repeated sequence typically comprises 34 amino acids, and the repeats are typically 91-100% homologous with each other. Polymorphism of the repeats is usually located at positions 12 and 13, and there appears to be a one-to-one correspondence between the identity of repeat variable-diresidues at positions 12 and 13 with the identity of the contiguous nucleotides in the TAL-effector's target sequence.

The TAL-effector DNA binding domain may be engineered to bind to a desired sequence, and fused to a nuclease domain, e.g., from a type II restriction endonuclease, typically a nonspecific cleavage domain from a type II restriction endonuclease such as Fokl (See, e.g., Kim et al. (1996) Proc. Natl. Acad. Sci. USA 93:1156-1160, which is incorporated by reference in its entirety herein, including any drawings). Other useful endonucleases may include, for example, Hhal, Hindlll, Nod, BbvCI, EcoRl, BglI, and AlwI. Thus, in preferred embodiments, the TALEN comprises a TAL effector domain comprising a plurality of TAL effector repeat sequences that, in combination, bind to a specific nucleotide sequence in a target DNA sequence, such that the TALEN cleaves the target DNA within or adjacent to the specific nucleotide sequence. TALENS useful for the methods provided herein include those described in WO10/079430 and U.S. Patent Application Publication No. 2011/0145940, which is incorporated by reference herein, including any drawings.

In some embodiments, the one or more of the nucleases is a zinc-finger nuclease (ZFN). ZFNs are engineered break inducing agents comprised of a zinc finger DNA binding domain and a break inducing agent domain. Engineered ZFNs consist of two zinc finger arrays (ZFA) each of which is fused to a single subunit of a non-specific endonuclease, such as the nuclease domain from the Fokl enzyme, which becomes active upon dimerization.

Useful zinc-finger nucleases include those that are known and those that are engineered to have specificity for one or more sites. Zinc finger domains are amenable for designing polypeptides that specifically bind a selected polynucleotide recognition sequence. Thus, they are amenable to modifying or regulating expression by targeting particular genes.

The activity of an enzyme or one or more targets or one or more genes native to the cell can be modified in a number of other ways, including, but not limited to, gene silencing or any other form of genetic modification, expressing a modified form of the enzyme or one or more targets that exhibits increased or decreased solubility in the cell, expressing an altered form of the enzyme or one or more targets that lacks a domain through which the activity of the enzyme is inhibited, expressing a modified form of the enzyme or one or more targets that has a higher or lower Kcat or a lower or higher Km for a substrate, or expressing an altered form of the enzyme or one or more targets or protein product of the one or more genes native to the cell that is more or less affected by feed-back or feed-forward regulation by another molecule in the pathway.

It will be recognized by one skilled in the art that absolute identity to the targets is not strictly necessary. For example, changes in a particular gene or polynucleotide comprising a sequence encoding a target or an enzyme can be performed and screened for activity. Typically, such changes comprise conservative mutations and silent mutations. Such modified or mutated polynucleotides and polypeptides can be screened for expression or function using methods known in the art.

Those of skill in the art will recognize that, due to the degenerate nature of the genetic code, a variety of polynucleotides differing in their nucleotide sequences can be used to encode a given enzyme or one or more targets of the disclosure. Due to the inherent degeneracy of the genetic code, other polynucleotides that encode substantially the same or functionally equivalent polypeptides can also be used. The disclosure includes polynucleotides of any sequence that encode the amino acid sequences of the enzymes or one or more targets utilized in the methods of the disclosure.

In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as the modified or variant polypeptides have an activity that is identical or similar to the referenced polypeptide. Accordingly, the amino acid sequence set forth in SEQ ID NO: 1 merely illustrates embodiments of the disclosure.

The disclosure also includes one or more polypeptides with different amino acid sequences than the specific proteins described herein if the modified or variant polypeptides have an activity that is desirable yet different from referenced polypeptide. In some embodiments, an enzyme may be altered by modifying the gene that encodes the enzyme so that the expressed protein is more or less active than the wild type version.

As an example, the expressed MMSET protein may be more or less active according to substitutions that could create a catalytically active MMSET, hyperactive MMSET, a catalytically dead MMSET, or any version in between. Table 1 shows specific amino acid substitution in MMSET (numbered according to SEQ ID NO: 1) and respective consequences.

TABLE 1

| MMSET mutation | Reported effect |
|---|---|
| F1177A | Hyperactive (in vivo) |
| Y1118A | Catalytic dead (in vivo) |
| Y1179A | Catalytic dead (in vitro, in vivo) |
| Y1092A | Catalytic dead (in vitro, in vivo) |

As will be understood by those of skill in the art, it can be advantageous to modify a coding sequence to enhance expression in a particular host, such as, without limitation, a yeast cell. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.”

Optimized coding sequences containing codons preferred by a particular prokaryotic or eukaryotic host (See, for example, Murray et al., 1989, Nucl Acids Res. 17: 477-508, which is incorporated by reference in its entirety herein, including any drawings) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively.

In addition, homologs of enzymes or the one or more targets useful for the compositions and methods provided herein are encompassed by the disclosure. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences.

It is recognized that residue positions that are not identical often differ by conservative amino acid substitutions. In cases where two or more amino acid sequences differ from each other by conservative substitutions, the percent sequence identity or degree of homology may practically be adjusted upwards to correct for the conservative nature of the substitution. Means for making this adjustment are well known to those of skill in the art (See, e.g., Pearson W. R., 1994, Methods in Mol Biol 25: 365-89, which is incorporated by reference in its entirety herein, including any drawings).

Sequence homology and sequence identity for polypeptides is typically measured using sequence analysis software. A typical algorithm used comparing a molecule sequence to a database containing a large number of sequences from different organisms is the computer program BLAST. When searching a database containing sequences from a large number of different organisms, it is typical to compare amino acid sequences.

Furthermore, any of the one or more genes native to the cell or genes encoding the enzymes or one or more targets or genes native to the cell (or any others mentioned herein (or any of the regulatory elements that control or modulate expression thereof)) may be optimized by genetic/protein engineering techniques, such as directed evolution or rational mutagenesis, which are known to those of ordinary skill in the art. Such action allows those of ordinary skill in the art to optimize the enzymes for expression and activity in yeast, bacteria, or any other suitable cell or organism.

For example, amino acid sequence variants of the protein(s) can be prepared by mutations in the DNA. Methods for mutagenesis and nucleotide sequence alterations include, for example, Kunkel, (1985) Proc Natl Acad Sci USA 82:488-92; Kunkel, et al., (1987) Meth Enzymol 154:367-82; U.S. Pat. No. 4,873,192; Walker and Gaastra, eds. (1983) Techniques in Molecular Biology (MacMillan Publishing Company, New York) and the references cited therein. Guidance regarding amino acid substitutions not likely to affect biological activity of the protein is found, for example, in the model of Dayhoff, et al., (1978) Atlas of Protein Sequence and Structure (Natl Biomed Res Found, Washington, D.C). Each of the above-cited references is incorporated by reference in its entirety herein, including any drawings.

In addition, genes encoding enzymes homologous to the one or more targets or enzymes can be identified from other fungal and bacterial species or other species if they are orthologous or if there is homology between the two chosen species. For example, a variety of organisms could serve as a source for any of the proteins described herein, including, but not limited to, Saccharomyces spp., including S. cerevisiae and S. uvarum, Kluyveromyces spp., including K. thermotolerans, K. lactis, and K. marxianus, Pichia spp., Hansenula spp., including H. polymorpha, Candida spp., Trichosporon spp., Yamadazyma spp., including Y. spp. stipitis, Torulaspora pretoriensis, Issatchenkia orientalis, Schizosaccharomyces spp., including S. pombe, Cryptococcus spp., Aspergillus spp., Neurospora spp., or Ustilago spp. Sources of genes from anaerobic fungi include, but are not limited to, Piromyces spp., Orpinomyces spp., or Neocallimastix spp. Sources of prokaryotic enzymes that are useful include, but are not limited to, Escherichia coli, Zymomonas mobilis, Staphylococcus aureus, Bacillus spp., Clostridium spp., Corynebacterium spp., Pseudomonas spp., Lactococcus spp., Enterobacter spp., and Salmonella spp.

Techniques known to those skilled in the art may be suitable to identify additional homologous genes and homologous enzymes. Generally, analogous genes and/or analogous enzymes can be identified by functional analysis and will have functional similarities. As an example, to identify homologous or analogous biosynthetic pathway genes, proteins, or enzymes, techniques may include, but are not limited to, cloning a gene by PCR using primers based on a published sequence of a gene/enzyme of interest or by degenerate PCR using degenerate primers designed to amplify a conserved region among a gene of interest.

Further, one skilled in the art can use other techniques to identify homologous or analogous genes, proteins, or enzymes with functional homology or similarity. Techniques include examining a cell or cell culture for the catalytic activity of an enzyme through in vitro enzyme assays for the activity (See, for example, Kiritani, K., Branched-Chain Amino Acids Methods Enzymology, 1970, which is incorporated by reference in its entirety herein, including any drawings), then isolating the enzyme with the activity through purification, determining the protein sequence of the enzyme through techniques such as Edman degradation, designing PCR primers to the likely nucleic acid sequence, amplifying the DNA sequence through PCR, and cloning the relevant nucleic acid sequence. To identify homologous or similar genes and/or homologous or similar proteins, analogous genes and/or analogous proteins, techniques also include comparison of data concerning a candidate gene or enzyme with databases such as BRENDA, KEGG, or MetaCYC. The candidate gene or proteins may be identified within the above-mentioned databases in accordance with the teachings herein.

### 7. Modification or Deletion of Native Genes

In some embodiments, the cell has a genetic modification and/or deletion of one or more genes native to the cell. Reduction or elimination of expression may occur through any method known to one skilled in the art and all ways of genetically modifying, deleting, and/or of reducing or eliminating expression of genes native to the cell are provided herein.

In particular, one skilled in the art will understand that any form of genetic alteration or genetic engineering or genetic modification, such as those set forth above related to expression, may be used as an alternative to deletion. In some embodiments, other forms of genetic modification that may be used as an alternative to deletion include, for example, without limitation, gene knockouts, mutation, gene targeting, homologous recombination, gene knockdown, gene silencing, gene addition, molecular cloning, gene attenuation, genome editing, or any technique that may be used to suppress or alter or enhance a particular phenotype.

In particular, one skilled in the art would understand that any form of genetic alteration or genetic modification or genetic engineering known to one skilled in the art with respect to the yeast genome would be particularly suitable (See, for example, Rothstein, R. J. (1983) Methods Enzymol 101, 202-211; Elledge, S. J., and Davis, R. W. (1988) Gene 70, 303-312; Cormack, B., and Castano, I. (2002) Methods Enzymol 350, 199-218; Rothstein, R. (1991) Methods Enzymol 194, 281-301; Wach, A., Brachat, A., Pohlmann, R., and Philippsen, P. (1994) Yeast 10, 1793-1808; Goldstein, A. L., and McCusker, J. H. (1999) Yeast 15, 1541-1553; Gueldener, U., Heinisch, J., Koehler, G. J., Voss, D., and Hegemann, J. H. (2002) Nucleic Acids Res 30, e23; Shoemaker, D. D., Lashkari, D. A., Morris, D., Mittmann, M., and Davis, R. W. (1996) Nat Genet 14, 450-456, each of which is incorporated by reference herein, including any drawings).

In some embodiments, genetic modification or deletion can occur when a cell is contacted with one or more nucleases capable of cleaving, i.e., causing a break at a designated region within a selected site as provided above. In some embodiments, the nuclease is a CRISPR/Cas-derived RNA-guided endonuclease. In some embodiments, the nuclease is a TAL-effector DNA binding domain-nuclease fusion protein (TALEN). In some embodiments, one or more of the nucleases is a zinc-finger nuclease (ZFN).

In some embodiments, the expression activity of the one or more genes native to the cell can be altered in a number of ways, including, but not limited to, expressing a modified form of a polypeptide where the modified form of the polypeptide exhibits increased or decreased solubility in the cell, expressing an altered form of a polypeptide that lacks a domain through which activity is inhibited, or expressing an altered form of a polypeptide that is more or less affected by feed-back or feed-forward regulation by another molecule in a pathway expressed in the cell. In some embodiments, the strength of a promoter, enhancer, or operator to which the nucleotide sequence for the one or more genes native to the cell is operably linked may also be manipulated, decreased, or increased or different promoters, enhancers, or operators may be introduced.

In some embodiments, genetic modification or deletion occurs by identifying genes through a candidate screening approach. Candididate genes are generally the genes with known biological function directly or indirectly regulating a process of a phenotype. In some embodiments, deletion occurs by one of the methods and techniques set forth above for expressing exogenous nucleic acids in cells.

As set forth in the examples, after the one or more exogenous nucleic acids encoding one or more targets is added to the cell, the orthologue of the one or more targets native to the cell is modified or deleted. In some embodiments, MMSET, or hyperactive MMSET, is added, and then SET2, the yeast orthologue of the MMSET gene, is deleted. In some embodiments, the modified and/or deleted one or more genes native to the cell comprises or consists of one or both of SET2 and LGE1.

### 8. Testing Catalytic Dead Mutants

To confirm that the one or more targets is required for the toxic phenotype, one can abrogate activity of the one or more targets using catalytically dead mutants to interact with the one or more targets. As set forth in the examples, catalytically dead mutants of MMSET were constructed to confirm MMSET activity was required for the toxic phenotype (See, Table 1).

In some embodiments, the method is able to distinguish between different degrees of partially inhibited MMSET. In some embodiments, the one or more targets comprises a mixture of hyperactive targets and/or catalytically dead targets, the hyperactive targets and/or catalytically dead targets varied in relative abundance to calibrate relative toxicity to the cell. In some embodiments, the mixture of hyperactive targets and/or catalytically dead targets comprises one or more MMSET proteins, each having at least one or more of the following mutations: F1177A, Y1118A, Y1179A, and/or Y1092A, wherein the residues are numbered according to SEQ ID NO: 1. In some embodiments, the catalytically dead mutants comprise MMSET-SET2 chimers.

### 9. Growing Cells Under Growth Conditions

The cells are grown under growth conditions. The method may be practiced with any growth conditions known to one skilled in the art for any type of cell. For each cell, there is a set of conditions, both physical and chemical, under which the cell can survive. Cells of different types have a variety of physical requirements for growth, including temperature, pH, nutrients, and stress. One skilled in the art would know how to vary these conditions for the type of cell.

Growth conditions may be exploited to make the respective cells grow at different rates and to increase differentiation between different cells of the assay. In some embodiments, growth conditions comprise omitting one or more nutrients. Which elements may be omitted or added would be well known to one skilled in the art. In some embodiments, the growth conditions omit one or more of histadine, uracil, and/or lysine.

In some embodiments, the growth conditions comprise growing the cell at a temperature of less than about 30° C. In some embodiments, the growth conditions comprise growing the cell at a temperature of less than about 29° C. In some embodiments, the growth conditions comprise growing the cell at a temperature of less than about 28° C. In some embodiments, the growth conditions comprise growing the cell at a temperature of less than about 27° C. In some embodiments, the growth conditions comprise growing the cell at a temperature of less than about 26° C. In some embodiments, the growth conditions comprise growing the cell at a temperature of less than about 25° C. In some embodiments, the growth conditions comprise growing the cell at a temperature of less than about 24° C. In some embodiments, the growth conditions comprise growing the cell at a temperature of less than about 23° C. In some embodiments, the growth conditions comprise growing the cell at a temperature of less than about 22° C. In some embodiments, the growth conditions comprise growing the cell at a temperature of less than about 21° C. In some embodiments, the growth conditions comprise growing the cell at a temperature of less than about 20° C.

### 10. Measuring Colony Sizes

In some embodiments, measuring growth of the cell comprises calculating colony size or population size. Measuring colony size may occur by any method known to one skilled in the art such as, for example, without limitation, observing and counting cells, measuring wet or dry mass, or measuring turbidity. Compound screens may utilize cells plated in 96 or 384 well plates to produce a visual phenotypic change in the cells that can be quantified. Cell phenotype may be measured as a viability assay. Cellular phenotype screens may also include, for example, without limitation, foci formation screens, nuclear and cellular morphology screens, and localization of proteins. Cell phenotype screens may also include, for example, without limitation, reporter gene assay screens.

In some embodiments, measuring growth of a cell comprises using a Z-factor. The Z-factor is often used to show the discriminatory power of a high throughput assay. In high throughput screens, experimenters often compare a large number (hundreds of thousands to tens of millions) of single measurements of unknown samples to positive and negative control samples. The Z-factor quantifies the suitability of a particular assay for use in a full-scale, high throughput screen.

A Z-factor is calculated using the equation

$$Z - factor = 1 - \frac{3 ( {σ}_{p} + {σ}_{n} )}{\vert {μ}_{p} - {μ}_{n} \vert} ,$$

where μ is the mean value, σ is the standard deviation, and p and n stand for the positive and negative controls, respectively.

In some embodiments, measuring colony sizes comprises using Hedge's effect. Hedge's effect is also used to show the discriminatory power of a high throughput assay. The Hedge's effect size, g, is calculated using the following formula:

$$g = \frac{{\overline{x}}_{1} - {\overline{x}}_{2}}{{s}^{*}} ,$$

where s* is the pooled standard deviation, which is calculated as:

$${s}^{*} = \sqrt{\frac{( {n}_{1} - 1 ) {s}_{1}^{2} + ( {n}_{2} - 1 ) {s}_{2}^{2}}{{n}_{1} + {n}_{2} - 2} .}$$

TABLE S
Sequences

| SEQ | | |
|---|---|---|
| ID | | |
| NO: | Region | Sequence |
| 1 | MMSET | MEFSIKQSPLSVQSVVKCIK |
| | | MKQAPEILGSANGKTPSCEV |
| | | NRECSVFLSKAQLSSSLQEG |
| | | VMQKFNGHDALPFIPADKLK |
| | | DLTSRVFNGEPGAHDAKLRF |
| | | ESQEMKGIGTPPNTTPIKNG |
| | | SPEIKLKITKTYMNGKPLFE |
| | | SSICGDSAADVSQSEENGQK |
| | | PENKARRNRKRSIKYDSLLE |
| | | QGLVEAALVSKISSPSDKKI |
| | | PAKKESCPNTGRDKDHLLKY |
| | | NVGDLVWSKVSGYPWWPCMV |
| | | SADPLLHSYTKLKGQKKSAR |
| | | QYHVQFFGDAPERAWIFEKS |
| | | LVAFEGEGQFEKLCQESAKQ |
| | | APTKAEKIKLLKPISGKLRA |
| | | QWEMGIVQAEEAASMSVEER |
| | | KAKFTFLYVGDQLHLNPQVA |
| | | KEAGIAAESLGEMAESSGVS |
| | | EEAAENPKSVREECIPMKRR |
| | | RRAKLCSSAETLESHPDIGK |
| | | STPQKTAEADPRRGVGSPPG |
| | | RKKTTVSMPRSRKGDAASQF |
| | | LVFCQKHRDEVVAEHPDASG |
| | | EEIEELLRSQWSLLSEKQRA |
| | | RYNTKFALVAPVQAEEDSGN |
| | | VNGKKRNHTKRIQDPTEDAE |
| | | AEDTPRKRLRTDKHSLRKRD |
| | | TITDKTARTSSYKAMEAASS |
| | | LKSQAATKNLSDACKPLKKR |
| | | NRASTAASSALGFSKSSSPS |
| | | ASLTENEVSDSPGDEPSESP |
| | | YESADETQTEVSVSSKKSER |
| | | GVTAKKEYVCQLCEKPGSLL |
| | | LCEGPCCGAFHLACLGLSRR |
| | | PEGRFTCSECASGIHSCFVC |
| | | KESKTDVKRCVVTQCGKFYH |
| | | EACVKKYPLTVFESRGFRCP |
| | | LHSCVSCHASNPSNPRPSKG |
| | | KMMRCVRCPVAYHSGDACLA |
| | | AGCSVIASNSIICTAHFTAR |
| | | KGKRHHAHVNVSWCFVCSKG |
| | | GSLLCCESCPAAFHPDCLNI |
| | | EMPDGSWFCNDCRAGKKLHF |
| | | QDIIWVKLGNYRWWPAEVCH |
| | | PKNVPPNIQKMKHEIGEFPV |
| | | FFFGSKDYYWTHQARVFPYM |
| | | EGDRGSRYQGVRGIGRVFKN |
| | | ALQEAEARFREIKLQREARE |
| | | TQESERKPPPYKHIKVNKPY |
| | | GKVQIYTADISEIPKCNCKP |
| | | TDENPCGFDSECLNRMLMFE |
| | | CHPQVCPAGEFCQNQCFTKR |
| | | QYPETKIIKTDGKGWGLVAK |
| | | RDIRKGEFVNEYVGELIDEE |
| | | ECMARIKHAHENDITHFYML |
| | | TIDKDRIIDAGPKGNYSRFM |
| | | NHSCQPNCETLKWTVNGDTR |
| | | VGLFAVCDIPAGTELTFNYN |
| | | LDCLGNEKTVCRCGASNCSG |
| | | FLGDRPKTSTTLSSEEKGKK |
| | | TKKKTRRRRAKGEGKRQSED |
| | | ECFRCGDGGQLVLCDRKFCT |
| | | KAYHLSCLGLGKRPFGKWEC |
| | | PWHHCDVCGKPSTSFCHLCP |
| | | NSFCKEHQDGTAFSCTPDGR |
| | | SYCCEHDLGAASVRSTKTEK |
| | | PPPEPGKPKGKRRRRRGWRR |
| | | VTEGK* |

### EXAMPLES

### Example 1: MMSET Toxicity in Yeast

The assay was enhanced by exacerbating the growth defect of the cell. Enhancement focused on lowering the growth rate of yeast strains expressing MMSET while maintaining viability, creating a synthetic sick variant as opposed to a synthetic lethal variant, as it were.

Mutant forms of MMSET were tested and it was shown that MMSET catalytic activity leads to a dramatic and quantifiable difference in colony size. A hyperactive mutant, F1177A (“MMSET-F”) was created, as well as several catalytically dead mutants, Y1118A, Y1179A, and Y1092A. Table 1 sets forth reported effects for mutant forms of MMSET, with the MMSET mutation provided on the left and the reported effect provided on the right. When expressed at high levels, both MMSET and MMSET containing a hyperactive mutation (MMSET-F) inhibit yeast cell growth. But, MMSET containing a catalytically dead mutation (Y1118A or “MMSET-Y”) did not. Similarly, larger colonies were produced using alternative catalytic dead MMSET mutations Y1092A or Y1179A.

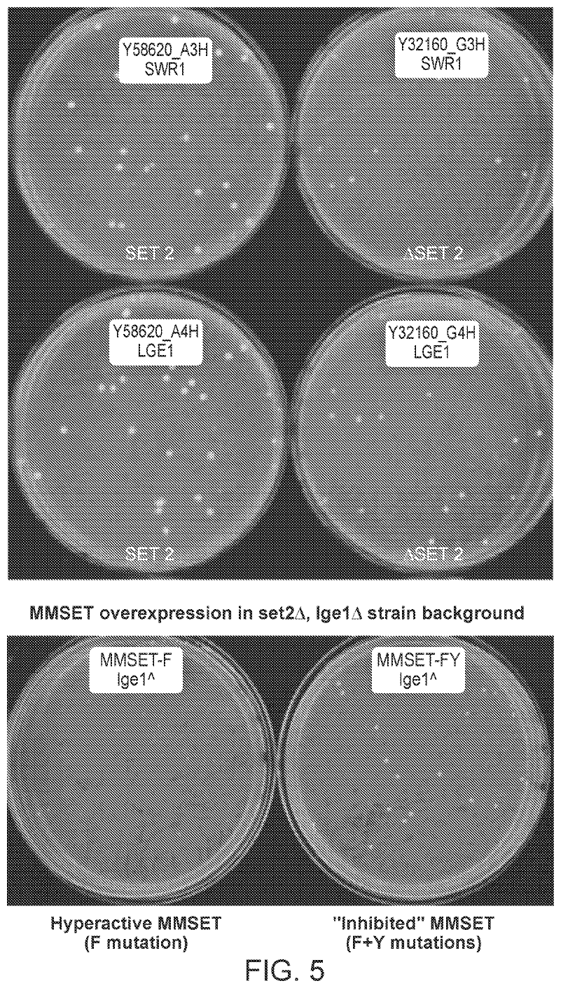

Hyperactive expression of MMSET was combined with gene deletions identified by large-scale testing of combinatorial gene deletions (See, for example, www.interactome-cmp.ucsfedu, which site is incorporated by reference herein in its entirety). In particular, deletion of LGE1 or SWR1 alone did not result in large changes in colony size, but when combined with a SET2 knockout, colonies were significantly smaller (See, FIG. 5, two panels on left). Expression of hyperactive MMSET in combination with SET2 and LGE1 deletion strains produced very slow-growing, small colonies (See, FIG. 5, drawing labelled “hyperactive MMSET (F mutation)”). When hyperactive MMSET-F was added to the strains, cell growth slowed even more (See, FIG. 5, right).

### Example 2: Modifying Growth Conditions Through Addition and/or Omission of Nutrients and Modification of the temperature

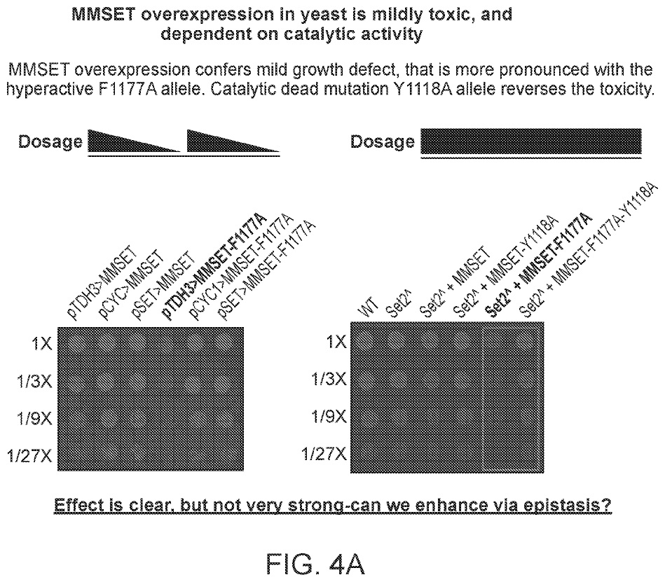

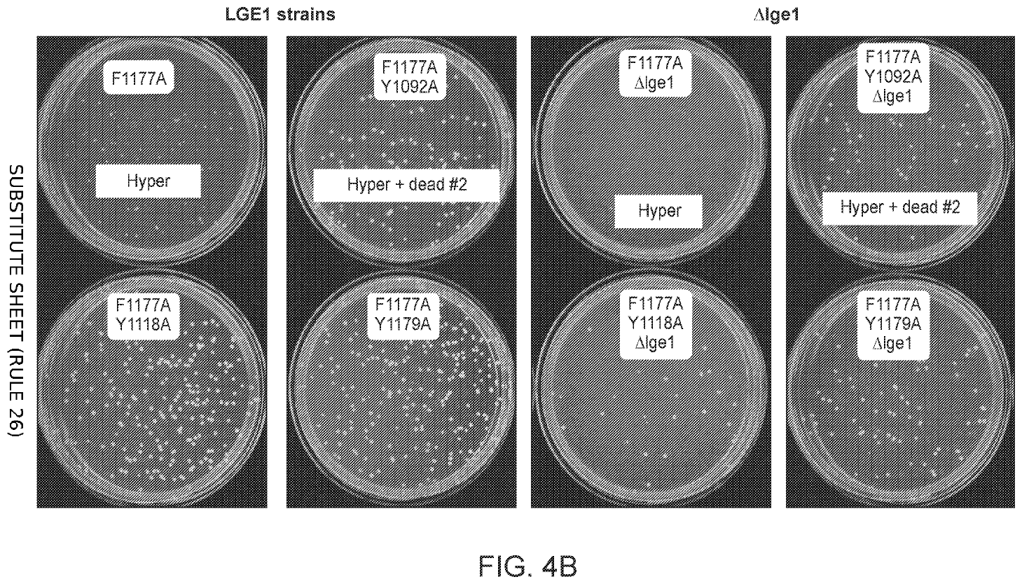

Differences in colony size were further amplified by choice of media and growth conditions. Each strain (MMSET-FY or MMSET-F in ASET2 AWE' background) was plated onto large-format complete synthetic media agar plates (24×24 cm) with several nutrients omitted (histadine, uracil, and lysine based on RNA-Seq results) and incubated at 30° C. for 3 days (FIG. 4). The plates were scanned and analyzed using custom software, and colony sizes were calculated based on fitted circles. Under these conditions, MMSET-FY colonies measure 11.04±1.04 pixels and MMSET-F colonies measured 2.01±0.75 pixels.

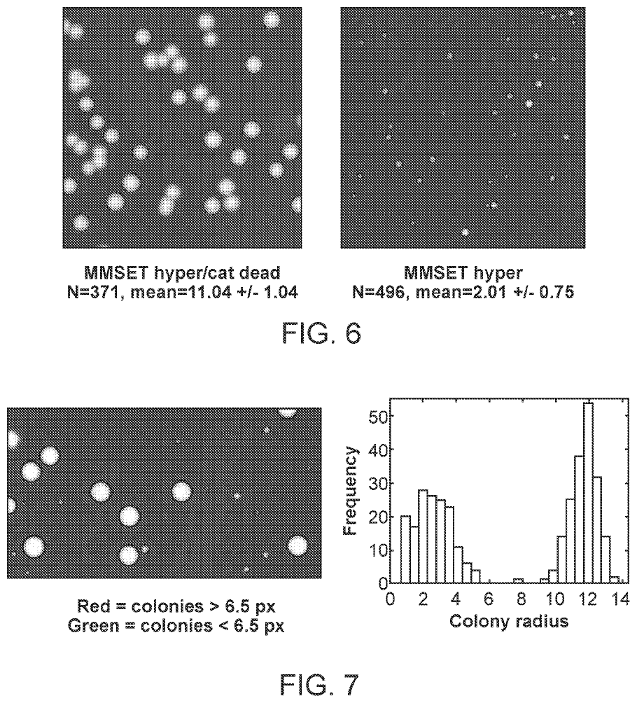

FIG. 6 shows that MMSET-FY (left) and MMSET-F (right) colonies display dramatically different colony sizes when plated on synthetic media that may omit at least one or more of histadine, uracil, and lysine.

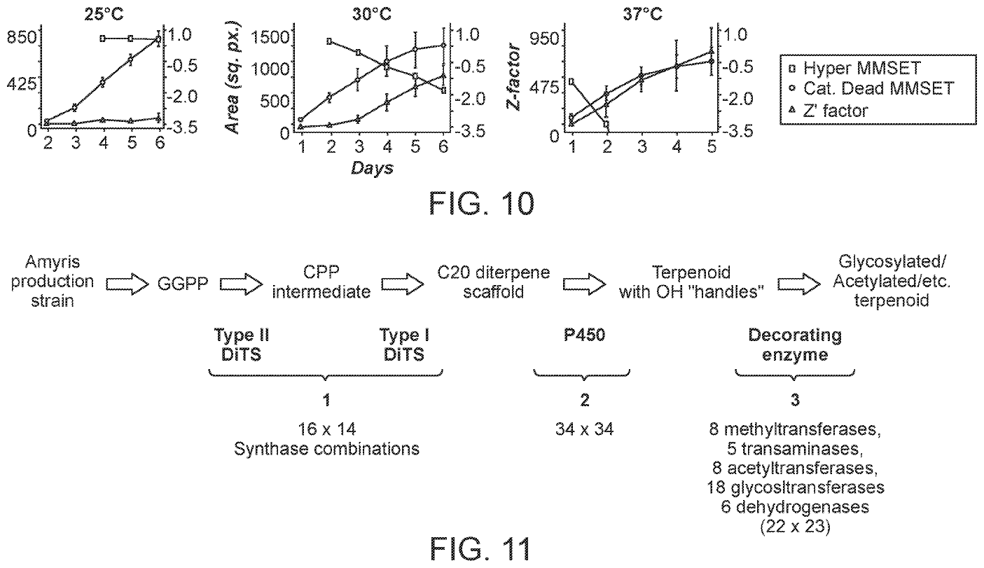

Additionally, lowering the incubation temperature led to an increased differentiation between hyperactive and catalytic dead MMSET strains (See, FIG. 10) with a Z′ of 0.7 (See, Example 3, below). FIG. 10 shows that incubation of the cells at 25° C. (left), 30° C. (middle), and 37° C. (right) resulted in an increased differentiation between hyperactive and catalytic dead mutants.

### Example 3: Measuring Assay Quality

An equal mixture of LGE1 knockout large (MMSET-FY) and small (MMSET-F) cells were plated on large-format agar plates at 30° C., plates were scanned, and resulting colony sizes were measured using custom software. Small colonies (less than 6.5 pixels in radius) were outlined and large colonies (greater than 6.5 pixels in radius) were also outlined (left).

From FIG. 7, a histogram of all measured colonies (right) shows clearly separated distributions for the two populations with no overlap. Small colonies were easily distinguished from large colonies by the software. Additionally, a separate program used by a colony-picking robot was also able to distinguish the two populations and would be able to pick large (MMSET inhibited) colonies preferentially.

A Z-factor of 0.405 was calculated using the equation. A Z-factor of at least 0.5 is ideal for a high throughput assay.

The Hedge's effect was calculated as 10.02.

### Example 4: Varying Fractions of Inhibited MMSET to Distinguish Between Different Degrees of Partially Inhibited MMSET

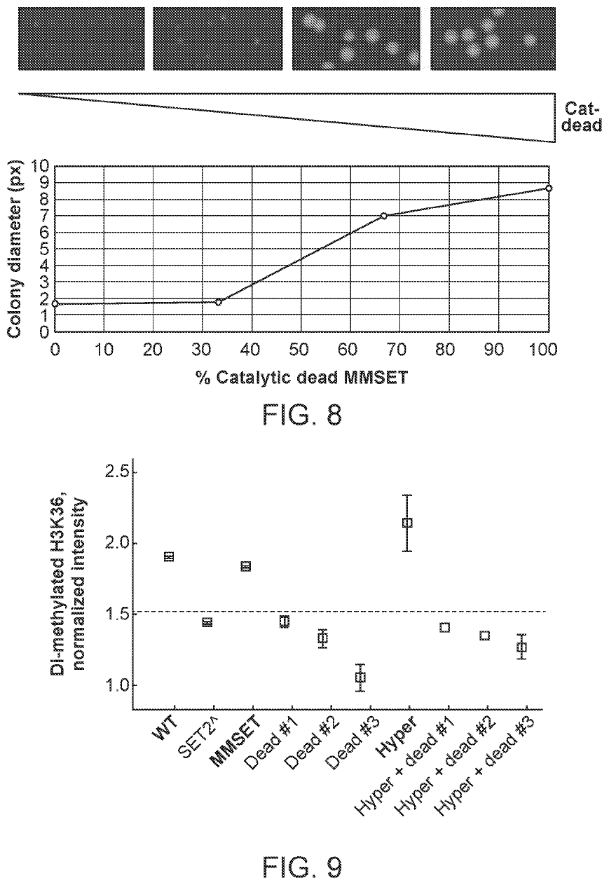

The assay was also tuned to be able to identify partially inhibited MMSET. Several yeast strains expressing a mixture of hyperactive and catalytically dead MMSET in a ΔLGE1 background, varying their relative abundance but maintaining a constant level of total MMSET, were made. Using the same software as above, colony sizes were measured and it was determined that colonies with inhibited MMSET were larger than those with 100% hyperactive MMSET. As shown in FIG. 8 and Table 2, cells with inhibited MMSET from 3 different catalytically dead mutants produce larger colonies in a ΔLGE1 background.

TABLE 2

| Category | Mutation | Colony width (px) | Stdev |
|---|---|---|---|
| Hyper | F1177A | 4.97 | ±1.13 |
| hyper + dead #1 | F1177A | 13.74 | ±4.61 |
| | Y1118A | | |
| hyper + dead #2 | F1177A | 15.63 | ±2.16 |
| | Y1092A | | |
| hyper + dead #3 | F1177A | 15.42 | ±2.29 |
| | Y1179A | | |

### Example 5: Dot Blot Verification of MMSET Activity in Yeast

Dot blots were performed to test MMSET activity. Dimethylation at Lys-36 on histone H3 (H3K36me2) is associated with actively transcribed genes. Histone methylation at Lysine 36 of histone 3 for wild-type MMSET, hyperactive MMSET, and catalytically dead mutants of MMSET was therefore tested.

The strains in Table 3 were grown to saturation, bead beat for lysis, and the lysates were spotted onto nitrocellulose. Using antibodies specific for di-methylated H3K36, as well as total histone H3, the relative level of di-methylated H3 for each strain were stained and quantified. Fluorescence was quantified and di-methylated signal was normalized to total histone measurements. Table 3 shows genotype, expected phenotype, and category.

FIG. 9 depicts the actual results. Strains with active SET2 or MMSET displayed higher levels of H3K36me2, confirming the activity of wild-type and hyperactive MMSET in yeast. All strains expressing catalytic-dead MMSET showed reduced levels of methylation.

TABLE 3

| | Expected | |
|---|---|---|
| Genotype | phenotype | Category |
| Wild-type S. cerevisiae | Methylation | WT |
| SET2\hat{ } | No methylation | SET2\hat{ } |
| SET2\hat{ } + MMSET | Methylation | MMSET |
| SET2\hat{ } + MMSET Y1118A | No methylation | dead #1 |
| SET2\hat{ } + MMSET Y1092A | No methylation | dead #2 |
| SET2\hat{ } + MMSET Y1179A | No methylation | dead #3 |
| SET2\hat{ } + MMSET F1177A | Methylation | Hyper |
| SET2\hat{ } + MMSET F1177A, | No methylation | hyper + dead #1 |
| Y1118A | | |
| SET2\hat{ } + MMSET F1177A, | No methylation | hyper + dead #2 |
| Y1092A | | |
| SET2\hat{ } + MMSET F1177A, | No methylation | hyper + dead #3 |
| Y1179A | | |

### Example 6: Biosynthetic Library Design

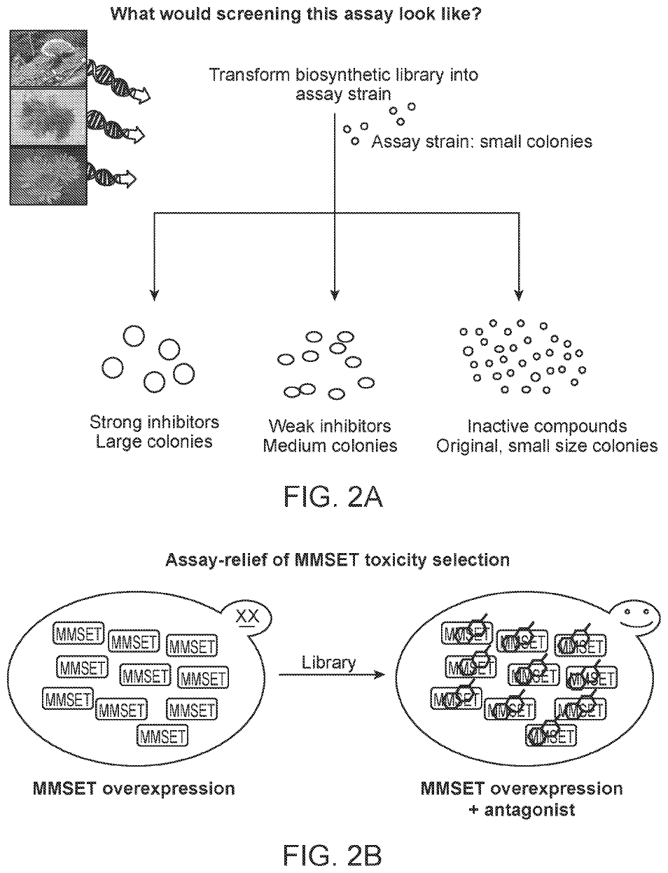

Biosynthetic libraries were transferred into assay strains to produce the natural or natural-like compound that could relieve toxicity in the method. High levels of MMSET slow yeast growth and a compound that inhibits MMSET activity will allow a yeast cell to grow faster (See, FIG. 2). For example, FIG. 2, bottom, left, shows MMSET overexpression and an unhappy cell; FIG. 2, bottom, right, shows MMSET overexpression and an antagonist of MMSET and a happy cell. The presence of strong inhibitors leads to strong colonies, weak inhibitors medium colonies, while inactive compound would lead to small colonies (See, for example, FIG. 2, top).

An actual biosynthetic library was constructed. The biosynthetic library contains terpene synthases, P450 monooxygenases and associated redox partners, and hydroxyl-modifying enzymes according to Table 4.

TABLE 4

| Uniprot ID | Name | Type |
|---|---|---|
| A0A075FAK4 | MvCPS1 | DiTS-II |
| A0A0M4MZ71 | TwTPS21 | DiTS-II |
| A0A0M4M0T9 | TwTPS28 | DiTS-II |
| Q675L4 | PaLAS | DiTS-II/I |
| E2IHE0 | CcCLS | DiTS-II |
| B8PQ84 | Sm.CPS-37 | DiTS-II |
| O22667 | SrCPS | DiTS-II |
| Q38802 | AtGA1 | DiTS-II |
| Q38802mut | AtGA1:H263Y | DiTS-II |
| X5A4D6 | CfTPS1 | DiTS-II |
| R9UNP0 | EpTPS7 | DiTS-II |
| Q6E7D7mut | OsCPS4:H501D | DiTS-II |
| G1DGI7 | SmCPSKSL1 | DiTS-II/I |
| N/A | Pleuro cyclase | DiTS-II |
| X4ZWN5 | CfTPS2 | DiTS-II |
| A0A075FA51 | MvCPS3 | DiTS-II |
| A0A0A7RRW2 | SpMils1_mut | DiTS-I |
| R9UPX6 | EpTPS1 | DiTS-I |
| Q6Z5J6 | OsKSL5j | DiTS-I |
| A4KAG8 | OsKSL6 | DiTS-I |
| C8XPS0 | Sm.KSL-46 | DiTS-I |
| A0A075FBG7 | MvELS | DiTS-I |
| X5A2Z7 | CfTPS3 | DiTS-I |
| A8M708 | SaDTS | DiTS-I |
| G9M5S4 | TaKSL1 | DiTS-I |
| Q9AJE4 | KgTS | DiTS-I |
| B9HI37 | PtKS | DiTS-I |
| A0A0M4M0A1 | EpTPS23 | DiTS-I |
| C8XPS0 | Sm.KSL-54 | DiTS-I |
| O65435 | TPS08 | TS |
| B5BSX1 | GuAO | P450 |
| N/A | P450_2v2_G | P450 |
| N/A | P450_1_G | P450 |
| J7I3T1 | CsKO4 | P450 |
| Q1PS23 | Aa.GB1_CYP71_idthigh | P450 |
| P54972 | AtKO | P450 |
| Q9K498 | SCO5223_CYP170A1 | P450 |
| P14779 | BM3_9-10A_F88A | P450 |
| O42713 | CYP76M8 | P450 |
| N/A | P450_3v2_G | P450 |
| Q50EK6 | PtAO_L466M | P450 |
| Q50EK6 | PtAO_LI23V | P450 |
| B5MEX6 | LsKO6 | P450 |
| Q7XZQ8 | PcFS | P450 |
| Q96330 | AtFO | P450 |
| Q9SWR5 | GmHFS | P450 |
| Q96WQ4 | TvCYP | P450 |
| P21334 | NcPS | P450 |
| F6H019 | VvCYP | P450 |
| H1A988 | GuAO | P450 |
| Q01332 | EvCO | P450 |
| B1NF20 | AmCS | P450 |
| Q4WLW7 | AfCYP | P450 |
| H2DH16 | PgDH | P450 |
| Q9Y7C8 | AtMH | P450 |
| N/A | CpPO3 | P450 |
| Q50EK6 | AbTO | P450 |
| O81346 | AtTO | P450 |
| Q6YTF1 | IpLO | P450 |
| P14779 | PcCO | P450 |
| Q9K498 | SmFS | P450 |
| Q6Q272 | AbPO | P450 |
| Q1PS23 | AtCH | P450 |
| Q6NKZ8 | AtKAH | P450 |
| Q9S818 | AtFO | P450 |
| N/A | P450_1_S | P450 |
| N/A | P450_1v2_G | P450 |
| N/A | P450_2_G | P450 |
| A0A142I6X1 | Sa.CYP816v8 | P450 |
| Q1PS23 | Aa.GB1_CYP71_idthigh | P450 |
| S4UX02 | CYP76AH1 | P450 |
| E5FA70 | PsCYP720B4 | P450 |
| P14779 | BM3_9-10A_F88V | P450 |
| N/A | P450_3_S | P450 |
| P14779 | BmP450_v1_24_AI_GB1 | P450 |
| Q50EK6 | PtAO_I223L | P450 |
| P14779 | BM3_9-10A_F88I | P450 |
| B1NF18 | PsSS | P450 |
| Q9FMA5 | AtBO | P450 |
| Q96323 | AtLO | P450 |
| Q6XAF4 | PsKO | P450 |
| Q4VCL5 | SrKO1 | P450 |
| Q7ZTS0 | DrBCO | P450 |
| Q6YTE2 | OsO | P450 |
| Q50EK6 | PtAH | P450 |
| H1A981 | MtAO | P450 |
| Q9HAY6 | DrBCO | P450 |
| Q50LH3 | EcSS | P450 |
| Q7XZQ6 | PcFO | P450 |
| O65815 | SrKAH | P450 |
| O04773 | CmFH | P450 |
| Q04468 | HtCO | P450 |
| P47195 | BsBS | P450 |
| P15101 | BtDO | P450 |
| Q05769 | MmPS | P450 |
| P0A110 | PpNO | P450 |
| Q6WG30 | TcTH | P450 |
| P11935 | AcDCS | P450 |
| Q7NWG4 | CvTA | ModEnz |
| Q9SPU3 | CbBAT | ModEnz |
| Q8S9G6 | TwTOAT | ModEnz |
| Q9FX01 | 3BETAHSD/D1 | ModEnz |
| A0A1C7D190 | BmTA | ModEnz |
| A0A067Z9B6 | AfMT | ModEnz |
| Q6ZD89 | OsFOMT | ModEnz |
| Q9F1Y5 | ScGMT | ModEnz |
| A0A077K7L1 | SbOMT2 | ModEnz |
| A0A077K9J6 | SbOMT1 | ModEnz |
| Q9M6E2 | TcBOAT | ModEnz |
| P00352 | retinal_dehydrogenase | ModEnz |
| K4D508 | UGT_SL2_4 | ModEnz |
| Q69TH5 | OsJ_UGT_v3 | ModEnz |
| A0A022QWN2 | EG_UGT_v1 | ModEnz |
| Q6VAA6 | UGT74G1 | ModEnz |
| Q6VAA8 | UGT91D_like3_1 | ModEnz |
| K4AME6 | Si_UGT40087 | ModEnz |
| Q6VAB4 | UGT76G1_R123_4 | ModEnz |
| J3LRY2 | Ob_UGT91B1_like | ModEnz |
| A0A0Q3JNI3 | Bd_UGT10840 | ModEnz |
| A8NTJ3 | SDR_G | ModEnz |
| N/A | ATF_G | ModEnz |
| K4D508 | UGT_SL2_mut_7 | ModEnz |
| Q6VAB0 | UGT85C2 | ModEnz |
| Q6VAB4 | UGT76G1 | ModEnz |
| O64988 | CbBAT | ModEnz |
| Q9ZTK5 | CrVOAT | ModEnz |
| Q9M6F0 | TcTOAT | ModEnz |
| Q9SPU3 | BEAT | ModEnz |
| O88451 | Retinol dehydrogenase 7 | ModEnz |
| P75214 | isopropanol_dehydrogenase_(NADP+) | ModEnz |
| Q2KNL6 | geraniol_dehydrogenase_(NADP+) | ModEnz |
| Q6VAB4 | UGT76G1_R3_V21_1 | ModEnz |
| Q0IP69 | OsNOMT | ModEnz |
| Q7WWK8 | AdTA | ModEnz |
| F2XBU9 | VfTA | ModEnz |
| B8AQD6 | eUGT11_5 | ModEnz |
| I1GP13 | Bd_UGT10850 | ModEnz |
| K4AA66 | Si_UGT35772 | ModEnz |
| B8AQD6 | On_UGT0GSF9 | ModEnz |
| F2DG34 | Hv_UGT_V1 | ModEnz |
| D7PI18 | PaMT | ModEnz |
| Q8GSN1 | CrFMT | ModEnz |
| C2ZAL1 | beta-alanine pyruvate_transaminase | ModEnz |

In Table 4, DiTS designates diterpene synthases of the indicated Type (I or II) and MondEnz designates hydroxyl-modifying enzymes. Library enzymes and corresponding amino-acid sequences were identified from literature searches, and DNA coding sequences were generated using codon optimization software for high-level expression in S. cerevisiae. In total, 30 terpene synthases, 68 P450s and 45 hydroxyl-modifying enzymes were included in the randomized library (See, Table 4). Expression constructs encoding these enzymes were integrated into the MMSET assay strain to test for MMSET inhibition (See, FIG. 11).

The platform strain was derived from an M2K background (Y33654) with 3 X-cutter landing pads at ALG1, YCT1, and MGA1 with additional GGPPS added (See, Table 5).

TABLE 5

| Strains | Name | \hat{ }LGE1? | \hat{ }SET2? | Description | Purpose |
|---|---|---|---|---|---|
| Y33654 | Grandparent | No | No | M2K background strain with | Grandparent |
| | | | | 3 X-cutter landing pads at | |
| | | | | ALG1, YCT1, MGA1 | |
| Y34026 | Parent | No | No | Low flux parent derived | Parent |
| | | | | from Y33654 with 1x | |
| | | | | Pgal10-Btris.GGPPS | |
| Y36240 | Production | No | No | Strain for testing library | Chemotype |
| | strain 1 | | | diversity (no MMSET). 3x | |
| | | | | CPRs (ATR1.v2, Aa.CPR, | |
| | | | | AfCPR.v2) @ GAS4 | |
| Y36242 | Production | No | No | Strain for testing library | Chemotype |
| | strain 2 | | | diversity (no MMSET). 3x | |
| | | | | CPRs (ATR1.v2, Aa.CPR, | |
| | | | | SaCPR3442V1) @ DIT1 | |
| Y39937 | Hyper 1 | Yes | Yes | Assay strain with hyperactive | MMSET |
| | | | | MMSET @SET2 locus and | assay |
| | | | | 3x CPRs @ GAS4 | background |
| Y39938 | Hyper 2 | Yes | Yes | Assay strain with hyperactive | MMSET |
| | | | | MMSET @SET2 and 3x | assay |
| | | | | CPRs @ DIT1 | background |
| Y39940 | Catdead 1 | Yes | Yes | Assay strain with | MMSET |
| | | | | catalytically dead MMSET | assay positive |
| | | | | @SET2 and 3x CPRs @ | control |
| | | | | GAS4 | |
| Y39941 | Catdead 2 | Yes | Yes | Assay strain with | MMSET |
| | | | | catalytically dead MMSET | assay positive |
| | | | | @SET2 and 3x CPRs @ | control |
| | | | | DIT1 | |
| Y40306 | Hyper 1 | No | | Assay strain with hyperactive | MMSET |
| | | | | MMSET @SET2 and 3x | assay |
| | | | | CPRs @ GAS4. These strains | background |
| | | | | have LGE1 gene intact to try | |
| | | | | to improve strain survival. | |
| Y40308 | Hyper 2 | No | | Assay strain with hyperactive | MMSET |
| | | | | MMSET @SET2 and 3x | assay |
| | | | | CPRs @ DIT1. These strains | background |
| | | | | have LGE1 gene intact to try | |
| | | | | to improve strain survival. | |
| Y40310 | Catdead 1 | No | | Assay strain with | MMSET |
| | | | | catalytically dead MMSET | assay positive |
| | | | | and 3x CPRs @ GAS4. | control |
| | | | | These strains have LGE1 | |
| | | | | gene intact to try to improve | |
| | | | | strain survival. | |
| Y40312 | Catdead 2 | No | | Assay strain with | MMSET |
| | | | | catalytically dead MMSET | assay positive |
| | | | | @SET2 and 3x CPRs @ | control |
| | | | | DIT1. These strains have | |
| | | | | LGE1 gene intact to try to | |
| | | | | improve strain survival. | |
| Y35892 | Hyper yellow | Yes | | Strain developed as in-plate | Size control |
| | | | | size control with hyperactive | (not used) |
| | | | | MMSET @SET2 and yellow | |
| | | | | carotenoid (2x Xd_CrtYB, | |
| | | | | 1x_XdCrtI). Strong | |
| | | | | carotenoid expression | |
| | | | | resulted in colonies that were | |
| | | | | smaller than the assay | |
| | | | | strains. | |
| Y35894 | Catdead yellow | Yes | | Strain developed as in-plate | Size control |
| | | | | size control with catdead | (not used) |
| | | | | MMSET @SET2 and yellow | |
| | | | | carotenoid (2x Xd_CrtYB, | |
| | | | | 1x_XdCrtI). Strong | |
| | | | | carotenoid expression | |
| | | | | resulted in colonies that were | |
| | | | | smaller than the assay | |
| | | | | strains. | |

Each of the enzymes was assigned a landing pad (P450S—ALG1, DiTS—YCT1, decorating enzymes—MGA1). Each enzyme type was directed to a specific locus by homologous flanking sequences, insuring that each strain received a full pathway complete with all categories of enzymes. This guarantees that each strain will express a coherent biosynthetic pathway. Within each locus, enzymes were randomly integrated. The number of potential genomic combinations resulting from this library is over 130 million. To allow for quality control, the library was also transformed into a yeast production strain without MMSET for genotypic and phenotypic analysis.

There is a large genomic potential to the full library and, in an ideal scenario, each transformation would sample at most 10,000 combinations. Accordingly, a smaller library that could be sampled more fully by each transformation was created (See, Table 6).

TABLE 6

| Uniprot | Name | Enzyme Type |
|---|---|---|
| A0A075FAK4 | MvCPS1 | DiTS-II |
| E2IHE0 | CcCLS | DiTS-II |
| B8PQ84 | Sm.CPS-37 | DiTS-II |
| Q38802 | AtGA1 | DiTS-II |
| Q675L4 | PaLAS | DiTS-II/I |
| A0A075FA51 | MvCPS3 | DiTS-II |
| A0A0A7RRW2 | SpMils1_mut | DiTS-I |
| C8XPS0 | Sm.KSL-46 | DiTS-I |
| A0A075FBG7 | MvELS | DiTS-I |
| X5A2Z7 | CfTPS3 | DiTS-I |
| A8M708 | SaDTS | DiTS-I |
| G9M5S4 | TaKSL1 | DiTS-I |
| Q1PS23 | Aa.GB1_CYP71_idthigh | P450 |
| P54972 | AtKO | P450 |
| P14779 | BM3_9-10A_F88A | P450 |
| N/A | P450_1_G | P450 |
| J7I3T1 | CsKO4 | P450 |
| A0A142I6X1 | Sa.CYP816v8 | P450 |
| S4UX02 | CYP76AH1 | P450 |
| N/A | P450_3_S | P450 |
| Q6XAF4 | PsKO | P450 |
| O65815 | SrKAH | P450 |
| Q7NWG4 | CvTA | Transaminase |
| A0A1C7D190 | BmTA | Transaminase |
| A0A077K9J6 | SbOMT1 | Methyltransferase |
| Q9SPU3 | CbBAT | Acetyltransferase |
| A8NTJ3 | SDR_G | Dehydrogenase |
| Q9M6F0 | TcTOAT | Acetyltransferase |
| Q6VAB4 | UGT76G1 | glycosyltransferase |
| D7PI18 | PaMT | Methyltransferase |
| Q7WWK8 | AdTA | Transaminase |
| F2XBU9 | VfTA | Transaminase |
| C2ZAL1 | beta-alanine--- | Transaminase |
| | pyruvate_transaminase | |

The smaller library consisted of 6 of each Type I and Type II DiTS, 10 P450s divided between two loci and 10 modifying enzymes (primarily transaminases) divided between two loci. The smaller library led to 22,500 potential genomic combinations.

Library colonies resulting from the MMSET assay strain transformation were subject to further genotyping and phenotyping by colony size to identify potential inhibitors. Library colonies resulting from production strain transformations were also analyzed for genotypic and phenotypic diversity to assess success in randomly sampling different genomic combinations and generating unique compounds. The production strains (See, Table 5) did not have MMSET or any of the epistatic LGE1/SET2 knockouts that may lead to inhibited growth.

### Example 7: Assessing Library Diversity in a Production Strain

Production strains (without MMSET) were transformed in parallel with the same DNA library as the MMSET assay strains. The colonies were genotyped by Next Generation Sequencing and phenotyped by GC-FID and UPLC-UV-CAD (Ultra Performance Liquid Chromatography-Ultraviolet-Charged Aerosol Detection). The measurements show that, without selection, genotypes are roughly randomly distributed and strains produce a variety of distinct, unique peaks in analytical assays.

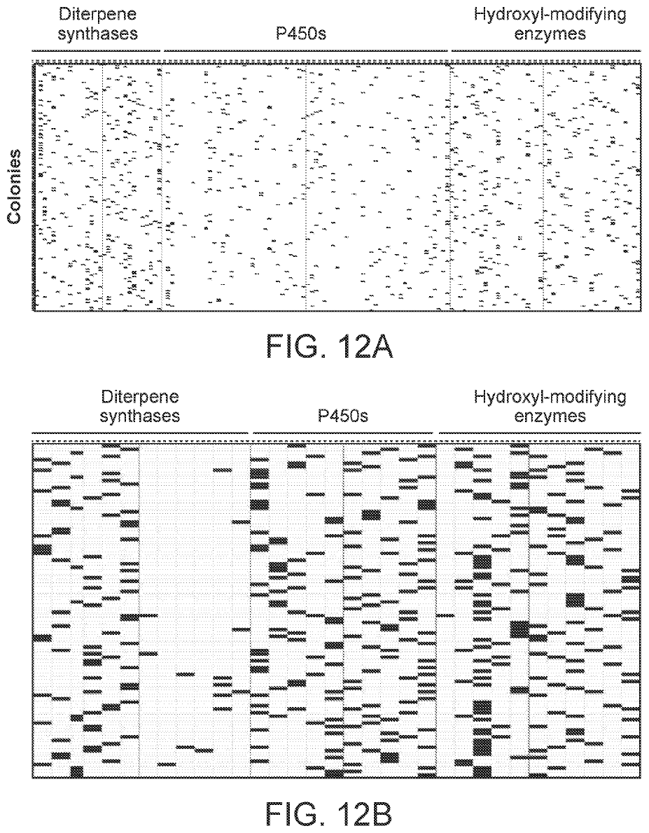

Sequencing was performed by lysing 192 colonies from the production strain library transformation and performing PCR to amplify each gene out of its genomic locus (6 PCRs per colony, one for each gene). All PCRs from the same colony were pooled into a single well for tagmentation and barcoding for Illumina paired-end sequencing. Following alignment of sequencing results, the enzyme integrated at each locus was identified (See, FIG. 12A, where genotypes are clustered by similarity). 191 of the 192 tested colonies had unique genotypes, demonstrating successful diverse sampling of genotype space in library transformation. The same type of analysis was carried out for the smaller library size as well (See, FIG. 12B).

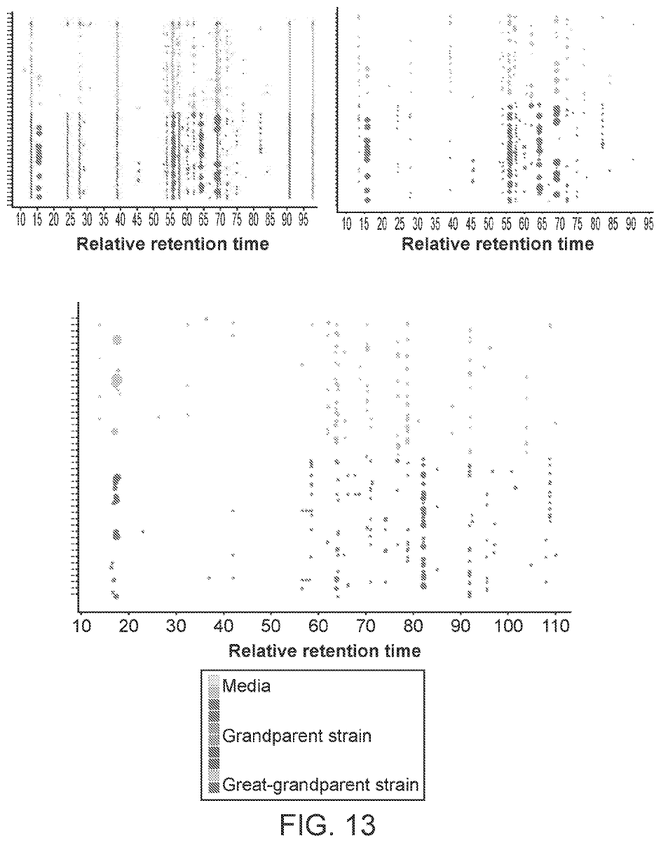

The same colonies were analyzed by GC-FID and UPLC-UV-CAD for phenotypic diversity, as measured through the appearance of novel peaks. Colonies from the production library strain were grown up in yeast production media and extracted with either methanol plus ethyl acetate for GC or ethanol and water for UPLC. A “dual column” GC method simultaneously injected each sample onto a nonpolar and a mid-polarity column, resulting in two chromatograms per colony (See, FIG. 13).

FIG. 13 shows chromatograms resulting from the nonpolar column (top) before background subtraction (left) and after (right). Chromatograms from the mid-polarity column (bottom) are shown after background subtraction. Each peak within a chromatogram is represented as a circle with size proportional to the peak area. Retention times are normalized to an internal standard. Parent, grandparent, and great-grandparent strains are shown in brown, blue, and orange respectively; media alone is shown in gray. 140 library colonies were tested and are shown in green. Light green colonies resulted from the small library with fewer enzymatic combinations and dark green points are from the full library transformation.

These chromatograms show the clear appearance of new and diverse peaks upon addition of library enzymes. UPLC traces were measured with three detectors: two UV (210 nm and 254 nm) and one CAD. These chromatograms similarly show many distinct novel peaks in library strains.

### Example 8: Quantifying Diversity in Library Strains

GC and UPLC chromatograms resulting from production colonies were analyzed using an automated peak calling and alignment algorithm. The algorithm identifies novel peaks from yeast production colonies by subtracting background peaks found in media and non-producing yeast. The algorithm identified 39 novel peaks by GC and 110 new peaks by UPLC in the 72 full library colonies tested by both methods. Similar numbers of new peaks were detected in the 72 small library colonies analyzed. By comparing chromatograms, it is evident that the two sample sets generated different compounds from each other. It is estimated that over 140 new compounds were generated in each set of 72 sampled colonies analyzed by both GC and UPLC.

### Example 9: MMSET Assay Strain Transformation and Screen Results

Seven transformations of the biosynthetic library into two different MMSET assay strain variants were completed (See, Table 7).

TABLE 7

| | | TRANSFORMATION | | |
|---|---|---|---|---|
| TRANSFORMATION | | EFFICIENCY | ASSAY | |
| NUMBER | LIBRARY | (TYPE) | STRAIN | TEMPERATURE |
| JL-1 | Full | Low (Electroporation) | LGE1\hat{ } | 25° C. |
| JL-2 | Small | Low (Electroporation) | LGE1\hat{ } | 25° C. |
| JL-3 | Small | High (Electroporation) | LGE1\hat{ } | 25°/30° C. |
| JL-4 | Full | High (Electroporation) | LGE1\hat{ } | 25°/30° C. |
| JL-5 | Full | High (Electroporation) | LGE1 | 25°/30° C. |
| | | | intact | |
| JL-6 | Full | High (Lithium acetate, | LGE1 | 25°/30° C. |
| | | chemical competence) | intact | |
| JL-7 | Full | High (LiAC) | LGE1\hat{ } | 25°/30° C. |

Hyperactive MMSET overexpressed and combined with SET2̂ and LGE1̂ was transformed by electroporation and grown at 25° C. The MMSET assay strain struggled to recover from the transformation, however, and few colonies were recovered (JL-1 to JL-1 from Table 7) from these first transformations.

Transformation was tested under more permissive conditions to mitigate the low efficiency, with both LGE1 intact in the MMSET assay strain, where the strain was grown at 30° C. and chemical transformation with lithium acetate (potentially gentler, and easier to scale) was used. Using these conditions, the library was further optimized and repeated insertion of the full library into the original MMSET assay strain was achieved.

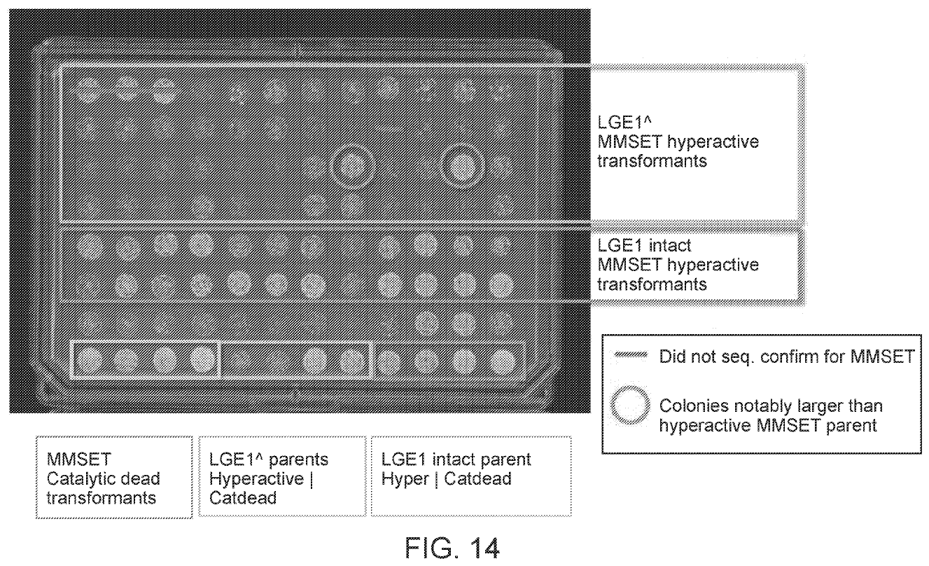

Library transformation plates were scanned daily starting when colonies became visible. Using image analysis software, colony sizes were quantified and labelled for picking. Chosen colonies were re-streaked onto fresh plates, presence of the WSET hyperactive allele was verified by colony PCR and Sanger sequencing, and strains were cultured in liquid media for storage and secondary colony size verification (See, FIG. 14). It is estimated that 3-4,000 unique genotypes were sampled producing over 2,000 compounds based on observed transformation.

FIG. 14 depicts colony size and growth rate verification for selected MMSET assay strains and their parents. Two MMSET assay strain variants were tested, LGE1 intact and LGE1̂. Chosen strains were cultured in liquid media, normalized by optical density, and spotted onto agar trays for colony size/growth rate verification and grown at 25° C. for four days before scanning. The bottom row of the agar plate shows catalytic dead and hyperactive MMSET control strains. The LGE1̂ MMSET assay strain (yellow box, bottom) displays clear differences between the hyperactive (left) and catalytic dead mutants (right). LGE1 intact parents are harder to distinguish by eye. In the top half of the plate, two colonies from the LGE1̂ MMSET assay strain appear to be faster growing strains. These strains were verified to contain hyperactive MMSET by sequencing.

Secondary colony size and growth rate verification on selected strains indicated two colonies with faster growing phenotypes than the hyperactive MMSET strain (See, FIG. 14, blue circles). For verification, strains were cultured in liquid media, normalized by optical density, spotted onto agar trays, and grown at 25° C. for four days before scanning. The bottom row of the agar plate shows catalytic dead and hyperactive MMSET control strains.

### Example 10: Verification of Growth Phenotypes in Potential Hits

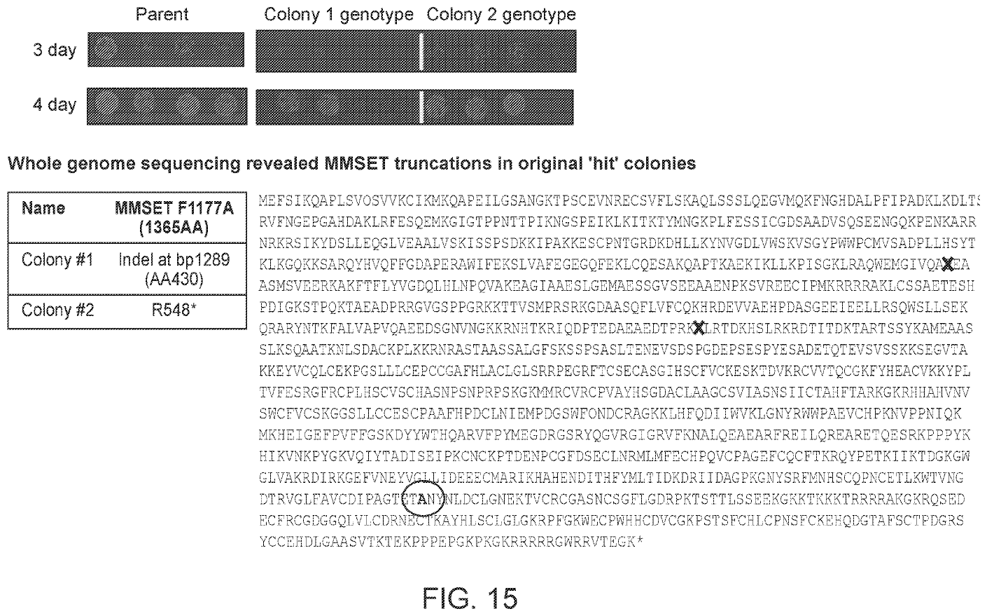

Two colonies with potentially inhibited MMSET were isolated from the library transformation (See, FIG. 15). When the selected biosynthetic pathways were re-transformed into the hyperactive MMSET assay strain, the faster growth phenotype was not recapitulated (See, FIG. 15). Whole genome sequencing of these strains revealed that the recovery of non-inhibited growth was due to premature truncation of the MMSET far upstream of the hyperactive allele resulting in loss of active MMSET expression. Therefore, the assay has proven capable of isolating colonies with inhibited MMSET, though in this case inhibition was genetic rather than chemical.

All publications and patent, applications cited in this specification are herein incorporated by reference as if each individual publication or patent application were specifically and individually indicated to be incorporated by reference. While the claimed subject matter has been described in terms of various embodiments, the skilled artisan will appreciate that various modifications, substitutions, omissions, and changes may be made without departing from the spirit thereof. Accordingly, it is intended that the scope of the subject matter limited solely by the scope of the following claims, including equivalents thereof

1. A cell comprising: i) one or more exogenous nucleic acids expressing one or more targets and ii) one or more genes native to the cell genetically modified and/or deleted, wherein the combination of the one or more targets with the genetic modification and/or deletion of one or more genes native to the cell is toxic to the cell.

2. The cell of claim 1, wherein genetic modifications and/or deletion of the one or more genes native to the cell provides a synthetic sick or synthetic lethal interaction to the cell.

3. The cell of claim 1, wherein the cell is a eukaryotic cell.

4. The cell claim 3, wherein the cell is a yeast cell, optionally wherein the yeast cell is Saccharomyces cerevisiae.

5. (canceled)

6. The cell of claim 1, wherein the one or more targets comprises a disease target, optionally wherein said disease target is a human disease target.

7. (canceled)

8. The cell of claim 7, wherein the disease target comprises or consists of MMSET.

9. The cell of claim 1, wherein the modified and/or deleted one or more genes native to the cell are selected from the group consisting of SET2, SWR1, and LGE1.

10. (canceled)

11. The cell of claim 1, further comprising one or more nucleic acids encoding enzymes that produce candidate inhibitor compounds.

12. The cell of claim 1, wherein the one or more targets comprises a mixture of hyperactive targets and/or catalytically dead targets, the hyperactive targets and/or catalytically dead targets varied in relative abundance to calibrate relative toxicity to the cell.

13. The cell of claim 12, wherein the mixture of hyperactive targets and/or catalytically dead targets comprises one or more MMSET proteins, each having at least one or more of the following mutations: F1177A, Y1118A, Y1179A, and/or Y1092A, wherein the residues are numbered according to SEQ ID NO: 1.

14. A method of detecting inhibitors of one or more targets, comprising:
a) providing a cell comprising one or more exogenous nucleic acids expressing the one or more targets;
b) genetically modifying and/or deleting one or more genes native to the cell, wherein the combination of the one or more targets with the genetic modification and/or deletion of the one or more genes native to the cell is toxic to the cell;
c) exposing the cell to candidate inhibitor compounds;
d) growing the cell under growth conditions; and
e) measuring growth of the cell,
wherein growth of the cell detects a candidate inhibitor compound as an inhibitor of the one or more targets.

15. The method of claim 14, wherein the combination of the one or more targets with genetic modification and/or deletion of the one or more genes native to the cell provides a synthetic sick or synthetic lethal interaction to the cell.

16. The method of claim 14, wherein the cell is a eukaryotic cell.

17. The method of claim 16, wherein the cell is a yeast cell, optionally wherein said yeast cell is Saccharomyces cerevisiae.

18. (canceled)

19. The method of claim 14, wherein the one or more targets comprises a disease target, optionally wherein said disease target is a human disease target.

20. (canceled)

21. The method of claim 20, wherein the disease target comprises or consists of MMSET.

22. The method of claim 14, wherein the modified and/or deleted one or more genes native to the cell are selected from the group consisting of SET2, SWR1, and LGE1.

23. (canceled)

24. The method of claim 14, wherein exposing the cell to candidate inhibitor compounds comprises expressing in the cell nucleic acids encoding enzymes that produce the candidate inhibitor compounds.

25. The method of claim 14, wherein exposing the cell to candidate inhibitor compounds comprises contacting the cell with the candidate inhibitor compounds.

26. The method of claim 25, wherein contacting the cell with candidate inhibitor compounds comprises adding the candidate inhibitor compounds to a cell culture.

27. The method of claim 14, wherein the growth conditions omit one or more of histidine, uracil, and/or lysine.

28. The method of claim 14, wherein the growth conditions comprise growing the cell at a temperature of less than about 30° C.

29. The method of claim 28, wherein the growth conditions comprise growing the cell at a temperature of less than about 25° C.

30. The method of claim 14, wherein measuring growth of the cell comprises calculating population size using a Z-factor or Hedge's effect.

31. The method of claim 14, wherein the one or more targets comprises a mixture of hyperactive targets and/or catalytically dead targets varied in relative abundance to calibrate toxicity to the cell.

32. The method of claim 31, wherein the mixture of hyperactive targets and/or catalytically dead targets comprises one or more MMSET proteins having at least one or more of the following mutations: F1177A, Y1118A, Y1179A, and/or Y1092A, wherein the residues are numbered according to SEQ ID NO: 1.
